(19) United States
(12) Patent Application Publication
(10) Pub. No.: US 2003/0158377 A1
(43) Pub. Date: Aug. 21, 2003

(54) Methods for preparation of macrocyclic molecules macrocyclic molecules prepared thereby and substrates and solid supports for use therein

(75) Inventors: Christopher Thomas Walsh, Wellesley, MA (US); Rahul Manu Kohli, Cambridge, MA (US); Michael D. Burkart, San Diego, CA (US); Martin D. Burke, Cambridge, MA (US)

Correspondence Address: EDWARDS & ANGELL, LLP, P.O. BOX 9169, BOSTON, MA, 02209, US

(73) Assignee: President and Fellows of Harvard College

(21) Appl. No.: 10/289,157

(22) Filed: Nov. 6, 2002

Related U.S. Application Data
(60) Provisional application No. 60/333,304, filed on Nov. 6, 2001.
(60) Provisional application No. 60/404,816, filed on Aug. 20, 2002.

(51) Int. Cl. C07K 1/04; C07H 17/08; C08G 63/48; C08G 63/91

(52) U.S. Cl. 530/333; 525/54.11; 525/351; 540/454; 536/7.1

(57) ABSTRACT

The invention features a new solid supports having at least one amino functionality and a linker bound to the solid support through at least a portion of the amino functional groups. The invention also features solid support bound substrates suitable for use in the formation of macrocycles by a TE domain catalyzed macrocyclization reaction. The invention features methods of making the solid supports and solid support bound substrates of the invention and methods of effecting macrocyclizations of solid support bound substrates. The invention further provides new macrocyclic molecules having one or more peptide domains and one or more polyketide domains in the macrocyclic ring. In another embodiment of the invention, libraries of macrocycles are provided as well as methods of forming libraries of macrocycles from libraries of solid support bound substrates are provided.

This application claims the benefit of U.S. Provisional Patent Application No. 60/333,304, filed on Nov. 6, 2001, and U.S. Provisional Patent Application No. 60/404,816 filed Aug. 20, 2002, each of which is incorporated by reference.

This invention was made with government support under Grants AI 10507-02 and GM-20011 from the National Institute of Health. The government has certain rights in the invention.

### FIELD OF INVENTION

The present invention relates to methods for the preparation of macrocyclic molecules and more particularly to macrocyclization of substrates bound to a solid-support catalyzed by an excised Type 1 thioesterase (TE) domain, and particularly to solid supports for the preparation of macrocyclic molecules and libraries of macrocyclic molecules that can be prepared using such supports and excised TE domains obviating traditional synthetic chemistry approaches to macrocyclic molecule synthesis.

### BACKGROUND OF THE INVENTION

An enormous range of medicinally important polyketide and peptide natural products assembled by modular polyketide synthases (PKSs), non-ribosomal peptide synthetases (NRPS) and mixed PKS/NRPS systems have macrocyclic structures, including the antibiotics erythromycin (PKS) and daptomycin (NRPS), the immunosuppressants cyclosporin (NRPS) and rapamycin (PKS/NRPS) and the antitumor agent epothilone (PKS/NRPS). PKSs and NRPSs are very large multifunctional proteins that are organized into sets of functional domains termed modules (Cane et al, Science (1998) 282:62-8; Marahiel et al, Chem. Rev. (1997) 97:2651-74). The sequence of modules corresponds directly to the structure of the product. Partially formed products are covalently tethered by thioester linkages to a carrier protein domain in each module. The thiol tether on each carrier domain is phosphopantetheine, which is attached to a conserved serine residue in the carrier protein in a post-translational priming reaction catalyzed by phosphopantetheinyl transferase (Lambalot et al, Chem. Biol. (1996) 3:923-36). Chain initiation involves loading a specific monomer onto each carrier protein's thiol tether. Subsequent chain elongation steps involve transfer of the growing chain from an upstream carrier protein to the adjacent downstream carrier protein-bound monomer. The full-length chain is almost always cyclized and released from the enzyme at the C-terminus of the NRPS or PKS system by a 28-35 kD TE domain (Cane et al, Science (1998) 282:62-8). During this final cyclization step, deacylation of the resulting acyl-O-TE intermediate at the C-terminal TE domain occurs either by intramolecular cyclization to form macrolactones or macrolactams or by hydrolysis.

The 6-deoxyerythronolide B synthase (DEBS) protein is a multidomain PKS protein with an integral TE domain that catalyzes cyclization of a protein-bound polyketide. Modification of domain identity or sequence in the natural DEBS protein by single or multiple domain substitutions or insertions of natural heterologous subunits generates DEBS protein variants that produce compounds with various ketide unit sequences. Systematic variation of the sequence of domains in the multidomain DEBS can in principle generate libraries of compounds (McDaniel et al, PNAS, (1999) 96:1846-51; McDaniel et al, Chem Biol, (2000) 7:77-84).

Kao disclosed the design and construction of engineered derivatives of the DEBS protein that is capable of synthesizing 6 and 8 member-ring lactones. The engineered DEBS derivatives included systems with protein modules, e.g. domains, exclusively from the DEBS system and hybrid derivatives that included protein modules from both the DEBS system and from the rapamycin PKS (RAPS) protein system. The DEBS-only derivative generated 6-member lactones and the DEBS-RAPS hybrid catalyzed the formation of a new 8-member lactone (Kao, J. Am. Chem. Soc. (1997) 119:11339-40).

The expression of a naturally occurring amino-terminal truncated form of a PKS protein to generate a macrocyclic molecule with smaller rings is described by Xue (Xue et al, Nature, (2000) 403:571-5). Truncation of the last condensation module from PikAIV in S. venezuelae leads to ‘skipping’ of the final condensation cycle in polyketide biosynthesis to generate a 12-membered ring macrolactone, 10-deoxymethynolide, instead of the 14-membered ring product molecoule, narbonolide.

Jacobsen et al disclosed a method for producing a series of polyketides by blocking the first condensation step of the DEBS protein system and introducing exogenous synthetic engineered molecules. The synthetic methods using the blocked DEBS protein system resulted in the highly selective production of a variety of polyketide molecules including aromatic and ring-expanded variants of 6-deoxyerythronolide B (Jacobsen et al, Science, (1997) 277:367-9).

The DNA sequence encoding the TE domain from 6-deoxyerythonolide B synthase (DEBS) has been excised and independently expressed and the domain isolated either as isolated TE domain enzyme (Gokhale, Chem Biol, (1999) 6:117-25) or as part of an ACP-TE di-domain protein (Aggarwal, J Chem Soc, Chem Comm, (1995) 15:1519-20). Thioester substrates were exclusively hydrolyzed to corresponding carboxylic acids by both the isolated TE domain and the ACP-TE didomain. The ACP-TE di-domain further hydrolyzes aryl esters. No cyclization was observed in these systems.

Many useful pharmaceuticals have macrocyclic structures (a large ring composed of 10 or more atoms). Traditional synthetic chemistry approaches to the synthesis of macrocyclic compounds have drawbacks including, but not limited to, low yields of macrocyclic molecule products, protecting groups required to block or mask reactive functionalities, and the need to carry out reactions in organic solvents.

International Publication No. WO 00/36093 describes a method for producing cyclic peptides and splicing intermediates of peptides in a looped conformation. The methods utilize the trans-splicing ability of split inteins to catalyze cyclization of peptides interposed between two portions of a split intein. The interaction of the two portions of the split intein creates a catalytically active intein, which catalyzes the formation and liberation of a cyclic peptide product.

Method of making libraries of macrocyclic compounds have been investigated to accelerate the drug discovery process. Several synthetic methods using traditional organic chemical synthesis techniques have been described in which libraries of synthetically-generated linear peptides have been prepared and cyclized using standard chemical synthesis followed by chemical cyclization of protected peptides (Spatola, et al. J. Med Chem. 39, 3842-3846 (1996); unprotected peptides cyclized using a metal-mediated cyclization technique (Zhang L, Tam J P JACS 121, 3311 (1999), or a metal-catalyzed cyclization process whereby a protected peptides having reactive functional groups is cyclized (Hiroshige M, et al JACS, 117, 11590 (1995) Alternatively an enzymatic cyclization of a linear peptide by a protease has been disclosed (Jackson D Y, et al JACS 117, 819 (1995)). In vivo production of cyclic peptides have also been shown using phage display (Wrighton N C, et al Science, 273 458 (1996) or intein cyclization (Scott C P, et al, Chem Biol. 8 801 (2001).

A poly(ethylene glycol acrylamide) resin (PEGA) has been reported including applications of in which a linker and peptide are bound to the resin for use in enzymatic assays (Renil M et al, J. peptide. Sci. 4, 195, (1998). PEGA has superior swelling properties in both organic and aqueous media (Meldal M, Tet Lett. 33, 3077 (1992).

The use of excised TE domains has been previously demonstrated in this lab (Kohli R M, et al., Biochemistry, 40, 7099 (2001); Trauger J W, et al., Biochemistry, 40 7092 (2001), Trauger J W, et al., Nature, 407, 215 (2000)). In particular, the tyrocidine, gramicidin, and surfactin TEs have been studied for general enzymatic activity and substrate specificity using shortened N-acetylcysteamine thioesters as linear substrates for TE domain catalyzed cyclization. These experiments demonstrated the promiscuity of the excised TE domains and the ability to insert non-natural peptides and vary the ring size from 6 to 16 amino acid residues. These previous studies have utilized N-acetylcysteamine thioester as the phosphopantetheine mimic, thus providing soluble TE substrates.

However, there remains an unfulfilled need for synthetic methods for preparing macrocyclic molecules in high yield without requiring functional group protection or carrying out reactions in organic solvents. It would be particularly desirable to develop a synthetic method of preparing linear macrocyclic molecule substrates onto a solid support and forming the macrocyclic molecule while cleaving the substrate from the solid support.

### SUMMARY OF THE INVENTION

The present invention provides new solid supports for preparing macrocyclic molecules from linear precursors and methods for making the same. The present invention further provides new macrocyclization substrates, particularly macrocyclization substrates bound to a solid support of the present invention for forming macrocyclic molecules such as cyclic peptides, cyclic polyketides, or macrocyclic molecules having peptide and polyketide domains and optionally one or more hydrocarbon or oxyalkylene groups. The present invention also provides a method for the cyclization of linear substrates bound to a solid support through a linker wherein macrocyclic ring-closure is effected preferably by the formation of an amide or an ester bond catalyzed by a thioesterase domain excised and expressed from the DNA sequence for non-ribosomal peptide synthetase (NRPS) or polyketide synthase (PKS) multidomain proteins.

The present invention also provides new substrates, particularly substrates bound to solid supports for preparing macrocyclic molecules having at least one peptide and at least one ketide domain and optionally one or more synthetic hydrocarbon or oxyalkylene groups from linear precursors and methods for making the same. The present invention also provides a method for the cyclization of linear substrates bound to a solid support through a linker wherein macrocyclic ring-closure is effected preferably by the formation of an amide or an ester bond catalyzed by a thioesterase domain excised and expressed from the DNA sequence for non-ribosomal peptide synthetase (NRPS) or polyketide synthase (PKS) multidomain proteins.

It is known that an integrally bound TE domain in a multidomain PKS or NRPS system catalyzes macrocyclization of protein-bound thioester substrates. However, applicants have discovered that excised TE domains can catalyze macrocycle formation using synthetic substrates. An enzymatic approach to macrocyclic molecule synthesis has advantages over traditional synthetic chemistry approaches including (i) high yield, (ii) regioselective cyclization that eliminates the need for protecting groups and (iii) reaction in aqueous systems.

The present invention provides new solid supports having a linker coupled thereto which mimics the enzyme-phosphopantetheine of the upstream domain in PKS or NRPS multi-domain systems which deliver a substrate to the TE domain for cyclization. Preferred solid supports of the invention include those solid supports suitable for solid phase synthesis according to Formula I:

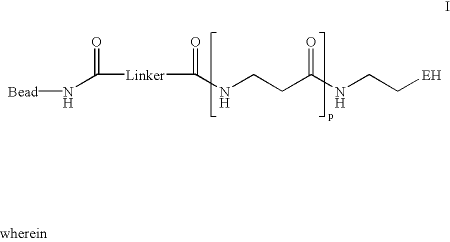

E is S or O;

p is an integer from 0-2;

Linker comprises a linear backbone disposed between the N-Polymer carbamoyl group and the —C(O)NH—(CH₂)₂—EH group, the linear backbone having between about 4 carbon atoms and about 20 carbon atoms and between about 0 and 10 hetero atoms selected from N, O or S, where each carbon of the linear backbone may be optionally substituted with 0, 1, or 2 groups selected from C₁₋₆alkyl, hydroxy, amino, halogen, C₁₋₆alkoxy, or oxo; and

Bead is a solid particle having amino functional groups to which a linker can be attached.

The present invention provides new substrates bound to a solid support having a linker coupled thereto which mimics the enzyme-phosphopantetheine of the upstream domain in PKS or NRPS multi-domain systems which deliver a substrate to the TE domain for cyclization.

The invention further provides methods of synthesizing solid supports according to Formula I, the method comprising the steps of

providing a solid or permeable particle having a plurality of amino functional groups;

contacting the bead with a carboxylic acid of the formula:

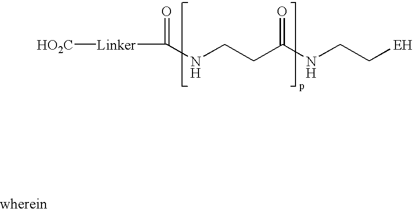

E is S or O; and

Linker comprises a linear backbone comprising between about 4 carbon atoms and about 20 carbon atoms and between about 0 and 10 hetero atoms selected from N, O or S, where each carbon of the linear backbone may be optionally substituted with 0, 1, or 2 groups selected from C₁₋₆alkyl, hydroxy, amino, halogen, C₁₋₆alkoxy, or oxo;

under conditions conducive to the formation of a solid support according to Formula I:

The present invention also provides solid support bound substrates which are bound to a solid support of Formula I or any sub-formula thereof, where the substrate is coupled to the solid support through the Linker. Solid-support bound substrates according to Formula V are suitable for use as macrocyclization substrates for TE domain catalyzed macrocyclization where the TE domain is a TE domain excised from a PKS or NRPS multi-domain system. Preferred substrate for TE domain catalyzed macrocyclization include solid support bound substrates according to Formula V:

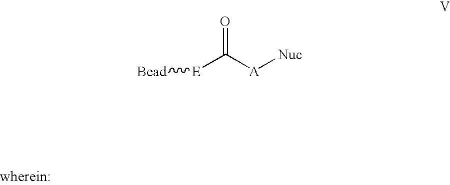

A is a peptidic sequence, synthetic hydrocarbon group, a polyketide, —(CH₂CH₂O)ₛ—, or a combination thereof having at least about 10 atoms in a linear backbone;

s is an integer from 1 to about 10;

Nuc is NH₂ or OH; and

Bead [symbol] E is a solid support according to Formula I or any subformulae thereof.

In other preferred solid support substrates of Formula V, the group A comprises at least one peptidic sequence, at least one polyketide sequence, and optionally one or more groups selected from a synthetic hydrocarbon group, —(CH₂CH₂O)ₛ—, or a combination thereof such that A has at least about 10 atoms in a linear backbone.

In another embodiment of the invention, a method of preparing solid support bound substrates according to Formula V where A is a linear peptidic sequence, the method comprising the steps of:

providing a solid support according to Formula I which comprises a solid particle which comprises a plurality of amino groups and at least one Linker coupled to the polymer through an amino group;

contacting the solid support having a Linker with a series of amino acid residues under conditions conducive to the formation of a specified amino acid sequence where a C-terminus of the sequence is coupled to the Linker to form a linear peptidic sequence bound to a solid support.

The invention further provides methods preparing a linear hybrid substrate having peptide and polyketide residues, the method comprising the steps of:

providing a solid support according to Formula I:

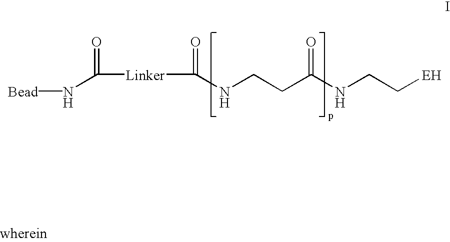

E is S or O;

p is an integer from 0-2;

Linker comprises a linear backbone having between about 4 carbon atoms and about 20 carbon atoms and between about 0 and 10 hetero atoms selected from N, O or S, where each carbon of the linear backbone may be optionally substituted with 0, 1, or 2 groups selected from C₁₋₆alkyl, hydroxy, amino, halogen, C₁₋₆alkoxy, or oxo; and

Bead is a solid particle having amino functional groups to which a linker can be attached;

contacting the solid support according to Formula A with a series of amino acid residues and polyketide residues under conditions conducive to the formation of a specified hybrid peptide/polyketide sequence coupled to the EH group of the Linker to form a linear hybrid substrate sequence bound to the polymer support.

The invention also provides a method for the preparation of macrocyclic molecules comprising:

providing a substrate comprising an activated acyl residue and a pendant nucleophile separated by a linear backbone where the activated acyl residue is coupled to a solid support according to Formula I:

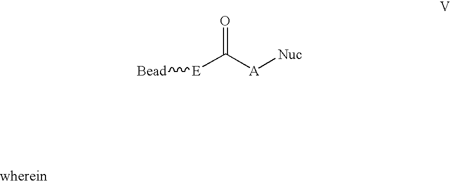

A is a peptidic sequence, synthetic hydrocarbon group, a polyketide, —(CH₂CH₂O)ₛ—, or a combination thereof having at least about 10 atoms in a linear backbone;

s is an integer from 1 to about 10;

Nuc is NH₂ or OH; and

Bead [symbol] E is a solid particle according to Formula I or a subformulae thereof; and

contacting a purified TE domain protein with the solid support bound substrate under conditions conducive to formation of an transient TE-O-acyl bond and subsequent formation of a macrocyclic product by displacement of a TE domain by a pendant nucleophile.

In preferred methods of preparing macrocyclic molecules provided by the present invention, the group A comprises at least one peptidic sequence, at least one polyketide sequence, and optionally one or more groups selected from a synthetic hydrocarbon group, —(CH₂CH₂O)ₛ—, or a combination thereof such that A has at least about 10 atoms in a linear backbone.

In preferred embodiments, the macrocyclization methods of the invention are carried out in an essentially aqueous medium that optionally includes one or more buffers and/or other organic or inorganic salts. Further, the buffered aqueous reaction medium preferably has a pH of about 5 to about 9, more preferably a pH of about 6 to about 8 and most preferably the reaction medium is essentially neutral with a pH of about 7. Preferred buffer additives include 3-(N-morpholino)propanesulfonic acid (MOPS) and other buffers that function well at or around neutral pH.

In preferred embodiments of the invention, the rate of the macrocyclization reaction catalyzed by an excised thioesterase domain protein is in the range of about 1 to about 100 macrocyclization reactions per minute per enzyme molecule. Useful amounts of macrocyclic compounds, e.g. about 1 μg or more of a macrocyclic compound, can be prepared with reaction times ranging from about 1 minute to about 120 minutes. The amount of hydrolysis byproduct is preferably less than the amount of the macrocyclization product, more preferably less than 50 wt % of the amount of the macrocyclization product molecule. In particularly preferred macrocyclization reactions catalyzed by an excised thioesterase domain protein, the amount of hydrolysis byproduct is less than about 25 wt % of the amount of the macrocyclization product molecule.

Preferred ring sizes of macrocyclic compounds produced by macrocyclization catalyzed by an excised thioesterase domain protein of the present invention comprise from about 12 to about 80 atoms. More specifically, for peptidic substrates of the invention preferred ring sizes comprise from 4 to about 24 amino acid residues, more preferably, from about 6 to 18 amino acid residues. Hybrid peptide/polyketide substrates and product peptide/polyketide macrocyclic molecules having a mixture of peptide domains and polyketide domains preferably comprise between about 4 and about 24 amino acid residues and between about 2 and about 25 ketide residues, more preferably between about 6 and 16 amino acid residues and between about 4 and about 12 ketide residues.

Preferably, macrocyclization substrates suitable for macrocyclization catalyzed by an excised thioesterase domain protein in accord with this invention are soluble in buffered or unbuffered aqueous solutions, or in aqueous solutions comprising a small amount, e.g. less than or equal to 20% v/v, of an organic solvent, at concentrations of at least about 0.1 gram of substrate per liter (g/L). Preferred organic solvents that are suitable for use in the present invention include sulfoxides, esters, amides and the like such as, e.g., dimethylformamide (DMF) and dimethylsulfoxide (DMSO).

Any of the solid-support bound substrates according to formula V or any subformula thereof or any solid support composition of Formula I or any of the subformulae thereof are suitable substrates and solid-supports for use in macrocyclization methods of the present invention.

Specific examples describe the use of TE domain protein excised from the Tyrocidine NRPS multidomain enzyme and from the surfactin synthetase multidomain enzyme to catalyze macrocyclization of substrates. However, the use of other excised TE domain proteins from other NRPS multidomain enzymes or from PKS multidomain enzymes that are appropriate to catalyze the macrocyclization of other substrates are also included in the scope of the present invention. The substrate specificity of other excised TE domain proteins can be determined by those skilled in the art by routine procedures analogous to the determination of substrate specificity for excised TycC TE domain protein disclosed herein. An appropriate excised TE domain protein can be chosen to catalyze the macrocyclization of a specified substrate based on structure commonalties between the specified substrate and the wild-type substrate of a particular TE domain protein. For example, excised TE domain proteins from PKS multidomain enzymes are preferable catalysts for the macrocyclization of polyketide substrates and excised TE domain proteins from NRPS multidomain enzymes are preferable for polypeptide substrates or substrates that comprise one or more peptide sequences.

In general, preparation of a library of compounds in which the end-group functionality, e.g., the functionality proximal to the nucleophile, Nuc, (e.g., XH where X is O or NH) of Formula V, is varied from the wild type substrate in order to determine substrate-TE domain specificity. Typically, the TE domain are significantly less sensitive to functional group variation at other positions in the substrate backbone. The ability of a TE domain to tolerate substrate variability may be probed by varying the functionality at any one residue or at a combination of residues concomitantly to determine the TE domain selectivity.

In specific embodiments of the present invention, the TE domain from tyrocidine NRPS (FIG. 2A), which as part of a multidomain NRPS enzyme catalyzes in nature the assembly of the cyclic decapeptide antibiotic tyrocidine A, can independently catalyze cyclization of solid support bound substrates according to Formula (VI) after excision from the multidomain enzyme system. The linker group can be, e.g., the nine C-terminal amino acid residues of the-natural tyrocidine A decapeptide substrate. Further acceptable substrate linkers can comprise depsipeptides (peptides in which one or more backbone amide bonds is replaced with an ester bond), a variable number of amino acid residues, synthetic non-peptidic spacers or a combination of one or more of the above groups, or the like. Additionally, substrates according to Formula (VI) where Nuc is OH also are cyclized by methods of the invention resulting in macrolactone formation.

The invention also provides a method to cyclize, catalyzed by the excised TE domain protein, substrates with a variable number of amino acid residues. For example, for the TE domain excised from the tyrocidine NRPS, solid-support bound substrates comprising at least 6 amino acid residues that include a key recognition end group residue are cyclized by the TE domain protein. Preferable substrates have between about 7 and about 16 amino acid residues.

The invention also provides a method for the macrocyclization of substrates wherein the macrocyclic ring formed can include both synthetic and biosynthetic amino acid residues, amino acid analogs, peptidomimetic components and one or more domains of non-peptidic, non-peptidomemetic linkers, and the like. Preferred substrates include (i) the N-terminal recognition residue which can vary depending on TE domain used for a macrocyclization reaction, and (ii) a C-terminal activated acyl group coupled to a solid-support through a linker according to Formula I or a subformulae thereof. For example, Type I TycC A TE domain generally requires a N-terminal amino acid recognition residue such as D-phenylalanyl or one of the residues listed in FIG. 4 or 5. The non-peptidic spacers comprise functional groups appropriate for formation of ester or amide bond linkages with optional peptide sequences, the N-terminal recognition residue or the C-terminal thioester activated acyl group. Preferably, the linker domains comprise functional groups that are sufficiently flexible to facilitate substrate macrocyclization by the methods of the present invention.

In other preferred embodiments of the present invention, the TE domain from tyrocidine NRPS (FIG. 2A) is suitable for cyclizing hybrid substrates having peptide and polyketide domains which are represented by Formula XII:

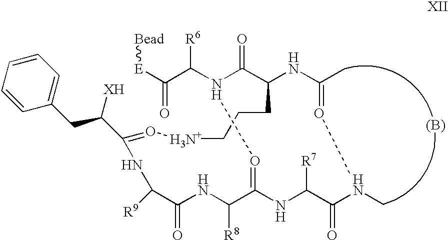

wherein R⁶, R⁷, R⁸, and R⁹ are independently selected from the group consisting of synthetic and biosynthetic amino acid residue side chains including side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol; and

X is O or NH.

Definitions

As used herein, the terms “excised thioesterase domain protein” or “excised TE domain protein” or “excised TE domain” refer to a protein domain normally present as the last domain in a large, multidomain polyketide synthase (PKS) or in non-ribosomal peptide synthetase (NRPS) proteins that normally catalyze in nature cyclization of a protein-bound thioester intermediate assembled by the upstream domains. For example, the term “excised TE domain protein” includes excised and expressed TycC TE from the tyrocidine NRPS (Trauger, Nature (2000) 407: 215-218) and also other Type I TE domain proteins in nature that are homologous to or provide function similar to the TE domain protein from the tyrocidine synthetase including gramicidin synthetase TE, surfactin synthetase TE, bacitracin synthetase TE, fengycin synthetase TE, calcium-dependent antibiotic (CDA) synthetase TE, microcystin synthetase TE, epothilone synthetase TE, daptomycin synthetase TE, syringomycin synthetase TE, nystatin synthetase TE, lichenysin synthetase TE, 6-deoxyerythronolide B synthase (DEBS), actinomycin synthetase TE, pristinamycin synthetase TE, virginiamycin synthetase TE, picromycin synthetase TE, and the like.

Excised TE domain protein also includes peptide sequences that are shorter than the complete, naturally occurring TE domain-containing NRPS or PKS protein but are longer than the TE domain peptide sequence, provided that the increased length of the peptide sequence does not prevent excised TE domain protein macrocyclization activity. Thus, the phrase “excised” refers to one or more domains of a multidomain protein system that have been isolated and expressed independently of the natural multidomain protein system. In practice, excised TE domain proteins generally are prepared by (i) isolating the part of the DNA that encodes the excised TE domain from the DNA encoding the TE-containing NRPS or PKS protein, (ii) expressing the DNA encoding the excised TE domain in a suitable expression host, e.g. in the bacterium Eschericia coli and (iii) purifying the expressed excised TE domain protein. Non-natural peptide sequences also can be included in the excised TE domain protein sequence to facilitate expression or purification of the excised TE domain protein. Typically, such excised TE domain proteins have a molecular weight less than about 100 kilodaltons (kD). For excised TE domain protein from a multidomain NRPS or PKS system that catalyzes substrate cyclization, preferred TE domain peptide sequences are in the range of about 27-35 kD.

As used herein, the phrases “key recognition residue” and “recognition residue” refer to the groups in a substrate that are necessary for macrocyclization to occur. In general, most key recognition residues are located near the portions of the substrate that react to form the macrocycle, e.g., near the N- and C-terminal ends of peptide substrates for the TE domain from the tyrocidine synthetase. In typical examples, the substrate groups near the nucleophile that reacts with the acyl-O-TE intermediate are key recognition residues that are necessary for TE domain catalyzed substrate macrocyclization to occur. Acceptable functional group varieation in the recognition residues may be determined by screening libraries of solid support substrates having diversity introduced at the proposed recognition residue and measuring the relative ratio of macrocyclization versus hydrolysis for each substrate of the library.

As used herein, the phrase “an amino acid side chain” refers to the distinguishing substituent attached to the α-carbon of an amino acid; such distinguishing groups are well known to those skilled in the art. For instance, for the amino acid glycine, the side chain is H; for the amino acid alanine, the side chain is CH₃, and so on.

As used herein, the term “amino acid” is intended to include common natural or synthetic amino acids and common derivatives thereof, known to those skilled in the art. Typical amino-acid symbols denote the L configuration unless otherwise indicated by a D appearing before the symbol.

As used herein, ketide residue refers to groups of the general formula, —CHR^{A}CHR^{B}— which may be coupled to form polyketides having two or more ketide residues. Typically, R^{A} and R^{B} are selected from hydrogen, hydroxy, keto, C₁₋₆alkyl, C₂₋₆alkenyl, C₂₋₆alkynyl, C₃₋₈cycloalkyl, C₇₋₁₂aralkyl, C₁₋₆alkoxy, and hydroxyC₁₋₆alkyl; or —CHR^{A}CHR^{B}— taken in combination forms a 1,2-vinylidene group. More preferably, R^{A} is hydrogen, methyl, ethyl, propyl or the like and R^{B} is hydroxy, C₁₋₆alkoxy or keto.

Ketide and polyketide building blocks suitable for use in the substrates of the present invention are functionalized with groups suitable for coupling to amino acid residues. Typically, ketide or polyketide building blocks comprise a carboxylate group at one end of the residue and an hydroxy or amino group at the other end of the residue. Particularly preferred are α,ω-amino acid functionalized polyketide residues such as polyketide residues of Formula IV such as ε-amino acids illustrated in FIG. 6.

The substrates herein described can have asymmetric centers or axes. All chiral, diastereomeric, and racemic forms are included in the present invention. Many geometric isomers of olefins and the like also can be present in the compounds described herein, and all such stable isomers are contemplated in the present invention.

The term “substituted”, as used herein, means that any one or more hydrogens on the designated atom is replaced with a group selected from the defined list, provided that the designated atom's normal valence is not exceeded, and that the substitution results in a stable compound. When a substituent is keto (i.e., =0), then 2 hydrogens on the atom are replaced. Keto substituents are not directly attached to aromatic ring atoms.

When any variable occurs more than one time in any constituent or formula for a compound, its definition at each occurrence is independent of its definition at every other occurrence. Thus, for example, if a group is shown to be substituted with 0-2 R*, then said group may optionally be substituted with up to two R* groups and R* at each occurrence is selected independently from the definition of R*. Also, combinations of substituents and/or variables are permissible provided that such combinations result in stable compounds.

As indicated herein, various substituents of the compounds of the present invention and various formulae set forth herein are “optionally substituted”, including, e.g., a linker or carboxylate leaving group. When substituted, those substituents can be substituted at one or more of any of the available positions, typically 1, 2, 3, 4, or 5 positions, by one or more suitable groups such as those disclosed herein.

Suitable groups or “substituted” moieties for hydrogen atoms in compounds of the invention include, e.g., halogen such as fluoro, chloro, bromo or iodo; cyano; hydroxyl; nitro; azido; alkanoyl, such as a C₁₋₆ alkanoyl group such as acyl and the like; carboxamido; alkyl groups including those groups having 1 to about 12 carbon atoms, preferably 1-6 carbon atoms; alkenyl and alkynyl groups including groups having one or more unsaturated linkages and from 2 to about 12 carbon atoms, preferably 2-6 carbon atoms; alkoxy groups including those having one or more oxygen linkages and from 1 to about 12 carbon atoms, preferably 1-6 carbon atoms; aryloxy groups such as phenoxy and benzyloxy; alkylthio groups including those moieties having one or more thioether linkages and from 1 to about 12 carbon atoms, preferably 1-6 carbon atoms; alkylsulfinyl groups including those moieties having one or more sulfinyl linkages and from 1 to about 12 carbon atoms, preferably 1-6 carbon atoms; alkylsulfonyl groups including those moieties having one or more sulfonyl linkages and from 1 to about 12 carbon atoms, preferably 1-6 carbon atoms; aminoalkyl groups such as groups having one or more N atoms and from 1 to about 12 carbon atoms, preferably 1-6 carbon atoms; carbocyclic aryl groups having 6 or more carbons, particularly phenyl and benzyl (e.g., wherein an Ar group can be substituted or unsubstituted biphenyl moiety); arylalkyl having 1 to 3 separate or fused rings and from 6 to about 18 carbon ring atoms, with benzyl being a preferred group; arylalkoxy having 1 to 3 separate or fused rings and from 6 to about 18 carbon ring atoms, with O-benzyl being a preferred group; or a heteroaromatic or heteroalicyclic group having 1 to 3 separate or fused rings with 3 to about 8 members per ring and one or more N, O or S atoms.

As used herein, “alkyl” is intended to include both branched and straight-chain saturated aliphatic hydrocarbon groups, having the specified number of carbon atoms. Examples of alkyl include, but are not limited to, methyl, ethyl, n-propyl, i-propyl, n-butyl, s-butyl, t-butyl, n-pentyl, and s-pentyl. Preferred alkyl groups are lower alkyl groups having from 1 to about 6 carbon atoms. The term C₁₋₆ alkyl as used herein means alkyl groups consisting of 1 to 6 carbon atoms, which may contain a cyclopropyl moiety.

“Cycloalkyl” is intended to include saturated ring groups, having a specified number of carbon atoms, such as cyclopropyl, cyclobutyl, cyclopentyl, or cyclohexyl and bridged or caged saturated ring groups such as norbornane or adamantane and the like. Preferred cycloalkyl groups are cycloalkyl groups having from 3 to about 8 ring atoms. The term C₃₋₈ cycloalkyl as used herein means cycloalkyl groups consisting of a aliphatic ring with 3 to 8 atoms in the ring.

“Alkenyl” is intended to include hydrocarbon chains of either a straight or branched configuration comprising one or more unsaturated carbon-carbon bonds, which may occur in any stable point. along the chain such as, e.g., ethenyl and propenyl. Preferred alkenyl groups are lower alkenyl groups having from 2 to about 6 carbon atoms. The term C₂₋₆ alkenyl as used herein means alkenyl groups consisting of 2 to 6 carbon atoms.

“Alkynyl” is intended to include hydrocarbon chains of either a straight or branched configuration comprising one or more triple carbon-carbon bonds that may occur in any stable point along the chain such as, e.g., ethynyl and propynyl. Preferred alkynyl groups are lower alkynyl groups having from 2 to about 6 carbon atoms. The term C₂₋₆ alkynyl as used herein means alkynyl groups consisting of 2 to 6 carbon atoms.

As used herein, the term “heterocyclic group” is intended to include saturated, partially unsaturated, or unsaturated (aromatic) groups having 1 to 3 (preferably fused) rings with 3 to about 8 members per ring at least one ring containing an atom selected from N, O or S. The nitrogen and sulfur heteroatoms may optionally be oxidized. The term or “heterocycloalkyl” is used to refer to saturated heterocyclic groups.

As used herein, the term “aryl” includes groups that contain 1 to 3 separate or fused rings and from 6 to about 18 ring atoms, without hetero atoms as ring members. Specifically preferred carbocyclic aryl groups include phenyl, and naphthyl including 1-napthyl and 2-naphthyl.

“Haloalkyl” is intended to include both branched and straight-chain saturated aliphatic hydrocarbon groups having the specified number of carbon atoms, substituted with 1 or more halogen (for example —Cᵥ(Xⁱ)_{wi}(H_{2v+1-Σ(wi)}) where v=1 to 6; Xⁱ=F(i=1), Cl(i=2), Br(i=3), I(i=4) and Σwᵢ≦2v+1). Examples of haloalkyl include, but are not limited to, trifluoromethyl, trichloromethyl, pentafluoroethyl, and pentachloroethyl. Preferred haloalkyl groups are lower halolkyl groups having from 1 to about 6 carbon atoms. The term C₁₋₆ haloalkyl as used herein means haloalkyl groups consisting of 1 to 6 carbon atoms.

As used herein, the term “hydrocarbon group” is intended to include alkyl, cycloalkyl, alkenyl, alkynyl, and aryl groups or a group that comprises a combination of two or more alkyl, cycloalkyl, alkenyl, alkynyl or aryl group regions. Hydrocarbon groups may further comprise heteroatoms such as N, O, F, Si, S, Cl, Br and the like. Preferably, hydrocarbon groups have from 0 to about 3 heteroatoms. The term lower hydrocarbon group as used herein means a hydrocarbon group consisting of 1 to 6 carbon atoms which may include 1, 2, or 3 heteroatoms.

As used herein, the term “lipophilic group” refers to any hydrophobic group that is soluble in or miscible with lipids, hydrocarbons and other hydrophobic materials. Examples of lipophilic groups include, but are not limited to, long-chain C₆-C₃₂ alkyl groups that include linear alkyls, branched alkyls with one or more branch points or linear or branched alkyls which include one or more C₃-C₈ cycloalkane groups, long-chain C₆-C₃₂ alkenyl groups with one or more C—C double bonds that include linear alkenyls, branched alkenyls with one or more branch points or linear or branched alkenyls which include one or more C₃-C₈ cycloalkane or cycloalkene groups, long-chain C₆-C₃₂ alkynyl groups with one or more C—C triple bonds that include linear alkynyls, branched alkynyls with one or more branch points or linear or branched alkynyls which include one or more C₃-C₈ cycloalkane groups or long-chain C₆-C₃₂ alkyl, alkenyl or alkynyl groups that are optionally substituted with aryl, halogen, alkoxy, mono- or di(C₁-C₆)amino, C₁-C₆-alkyl ester.

As used herein, the term “cyclic lipopeptide” refers to cyclic peptides or cyclic depsipeptides that include one or more lipophilic groups, as well as cyclic peptides or depsipeptides that include one or more non-peptidic groups and one or more lipophilic groups.

“Alkoxy” means an alkyl group as defined above with the indicated number of carbon atoms attached through an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Preferred alkoxy groups are lower alkoxy groups having from 1 to about 6 carbon atoms.

The term “halogen” means fluorine, chlorine, bromine, or iodine.

### BRIEF DESCRIPTION OF THE DRAWINGS

FIG. 1 is a systematic illustration of variation in the solid supports and solid support bound substrates of certain embodiments the invention;

FIG. 2 is a reaction scheme for the formation of Pantebead, a solid support in accord with one embodiment of the invention based upon poly(ethylene glycol acrylamide), PEGA;

FIG. 3 is a reaction scheme for the formation of an illustrative solid support bound substrate of one embodiment of the invention and an illustrative macrocyclization of a solid support bound substrate by a TE domain protein;

FIG. 4 is a is table listing the members of an illustrative library of solid support bound substrates of a decapeptide where each substrate has a different amino acid residue at the N-terminus;

FIG. 5 is a table listing relative rates of cyclization versus hydrolysis for a variety of solid support bound substrates of the library tabulated in FIG. 4

FIG. 6 is a synthetic scheme for the preparation of illustrative polyketide building blocks which are suitable for use in the substrates of the present invention;

FIG. 7 is a synthetic procedure for TE domain catalyzed macrocyclization of a hybrid substrate and a series of HPLC traces showing macrocyclization of single and double polyketide building block insertion into the macrocyclic ring system;

FIG. 8 is a comparison of natural polyketide substitution with synthetic ε-amino acid polyketide building blocks and two illustrative protective polyketide building blocks suitable for use in the preparation of substrates bound to a solid support; and

FIG. 9 is a macrocycle prepared from a substrate according to Formula XII.

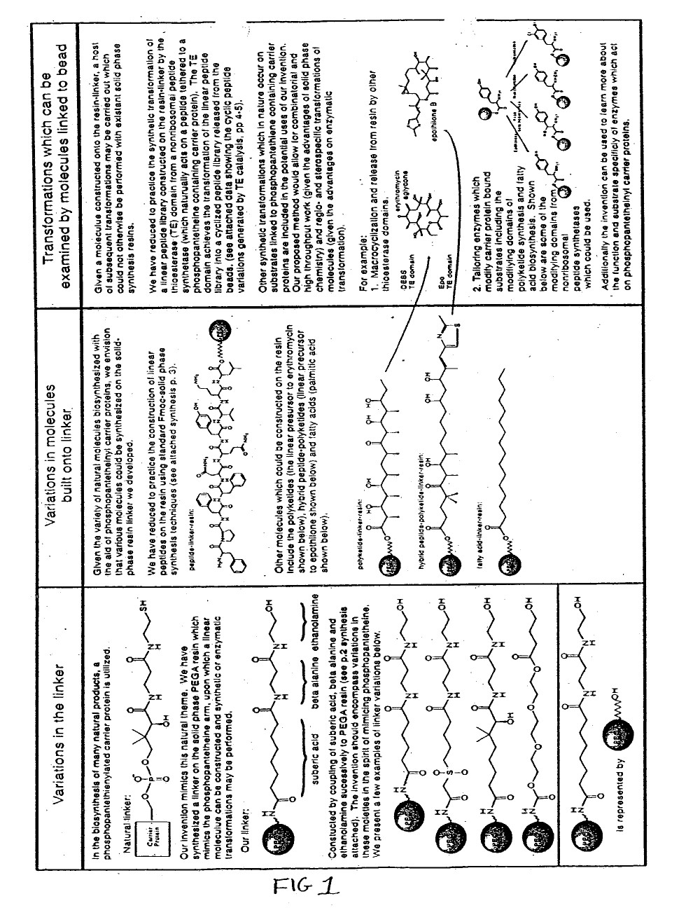

### DETAILED DESCRIPTION OF THE INVENTION INCLUDING PREFERRED EMBODIMENTS

The present invention provides new solid supports and methods for macrocyclic molecule synthesis that involves the use of an excised thioesterase (TE) domain protein from a non-ribosomal peptide synthetase (NRPS) or polyketide synthase (PKS) multidomain protein systems to catalyze the cyclization of synthetic substrates. They are useful for a wide variety of substrates, including substrates that differ from a wild-type TE domain substrate. The macrocyclization methods of the present invention are generally useful for the preparation of a wide range of macrocyclic molecules including pharmaceutical agents or libraries of macrocyclic molecules. Preferred solid support bound macrocyclic substrates of the invention include peptide, ketide and synthetic peptide/polyketide hybrid substrates having at least one peptide domain and at least one polyketide domain.

The present invention provides a novel solid support having amino groups for attaching linker groups. Preferred supports comprise a polymer, a graft copolymer or a polymer blend having amino groups distributed on the surface of the solid support, dispersed through out the polymer composition or a combination thereof. In certain preferred embodiments, insoluble polymers having amino groups capable of swelling in organic or aqueous media such as poly(ethylene glycol acrylamide), having linker groups coupled to one or more amino groups of the polymer support. In general, suitable linker groups of the invention function as phosphopantetheine mimics and as an attachment point for constructing substrate molecules on the polymer of the solid support. Preferred polymers having linker groups bound thereto swell in water sufficiently to allow diffusion of substrate synthons, such as amino acids, ketides, and the like that are used to construct substrates on the linker groups, macrocyclic product molecules formed by cleavage and subsequent cyclization of a substrate from the solid support, and TE domain enzymes throughout the polymer matrix.

In one embodiment of the invention, a solid support according to Formula I is provided.

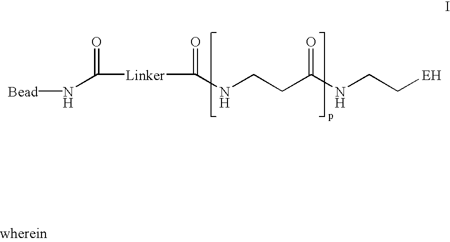

E is S or O;

p is an integer from 0-2;

Linker comprises a linear backbone disposed between the N-Polymer carbamoyl group and the —C(O)NH—(CH₂)₂—C(O)NH—(CH₂)₂—EH group, the linear backbone having has between 2 and 12 carbon atoms and from 0 to 6 heteroatoms selected from N, O and S in the linear backbone, where each carbon of the linear backbone may be optionally substituted with 0,1, or 2 groups selected from hydrogen, C₁₋₆alkyl, hydroxy, amino, halogen, C₁₋₆alkoxy, or oxo; and

Bead is a solid particle having amino functional groups to which a linker can be attached.

Preferred solid supports of the invention according to Formula I include solid supports, according to Formula II:

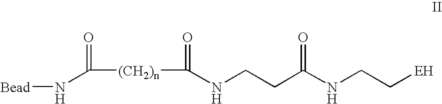

wherein n is an integer from about 3 to about 12.

Additional preferred solid supports of Formula I include those of Formula III:

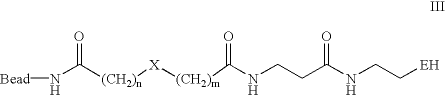

wherein n and m are independently selected integers from about 1 to about 10 m+n is about 3 to about 12; and

X is —CH(OH)—, —CH(CH₃)—, —C(CH₃)₂—, —S—, —O—, —S(O)—, or —S(O)₂.

More preferred substrates according to Formula III include those solid supports wherein

m and n are independently selected integers from about 1 to about 6;

m+n is from about 4 to about 8;

E is O; and

X is —S(O)— or —S(O)₂—.

Other preferred solid-supports of the invention include those solid supports according to Formula IV:

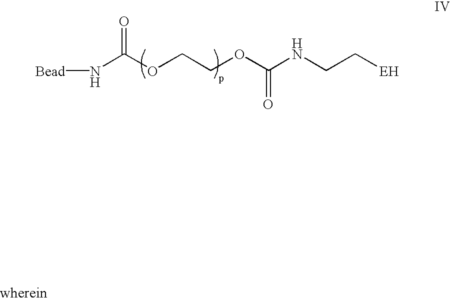

E is O or S;

p is an integer from about 1 to about 9; and

Bead is a solid particle having amino functional groups to which a linker can be attached.

Other preferred solid supports of the invention according to Formula IV include those solid supports in which one or more of the ethereal oxygen groups of the Linker are replaced by sulfur atoms to form a Linker group having thioether linkages or a combination of ether and thioether linkages.

Particularly preferred solid supports of the invention including those solid supports of any one of Formula I-IV include supports where E is O. Other particularly preferred solid supports of the invention include those where Bead comprises a water insoluble polymer having amino functional groups, where particularly preferred polymers swell in aqueous or organic solvents to allow solutes to diffuse or otherwise permeate through the polymer. Particularly preferred polymers having amino groups include polystyrene beads coated with aminated poly(ethylene glycol), e.g., Tentagel, and poly(ethylene glycol acrylamide), e.g., PEGA. PEGA isand controlled pore glass (CPG) are particularly preferred materials for use in any of the solid supports of solid support bound substrates of the invention.

Particularly preferred solid supports of the invention are used for solid-phase synthesis of a linear amino acid sequence, a linear polyketide sequence or a fatty acid residue, where the linear amino acid sequence, a linear polyketide sequence or a fatty acid residue is constructed on the support using standard solid phase synthetic techniques or previously synthesized groups are attached whole. Preferably, the linear amino acid sequence, a linear polyketide sequence or a fatty acid residue are attached to the solid support through the EH group pendant from the Linker. Most preferably solid supports can be recycled for repeated use where two or more linear amino acid sequence, a linear polyketide sequence or a fatty acid residue groups may be sequentially coupled to the solid support.

In another embodiment, the invention features methods of preparation of a solid support of Formula I, comprising:

providing a solid particle having a plurality of amino functional groups;

contacting the bead with a carboxylic acid of the formula:

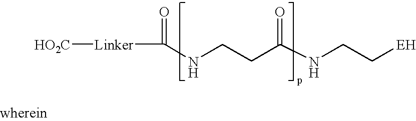

E is S or O;

p is an integer from 0-2; and

Linker comprises a linear backbone comprising between about 4 carbon atoms and about 20 carbon atoms and between about 0 and 10 hetero atoms selected from N, O or S, where each carbon of the linear backbone may be optionally substituted with 0, 1, or 2 groups selected from C₁₋₆alkyl, hydroxy, amino, halogen, C₁₋₆alkoxy, or oxo;

under conditions conducive to the formation of a solid support according to Formula I.

In other methods of preparing solid supports of Formula I, the method comprises steps of

providing Bead having a plurality of amino functional groups;

contacting the Bead with one or more chemical building blocks under conditions conducive to the formation a solid support having a group bound to an amino group of the solid support of Formula I.

In a preferred method of preparing a solid support of Formula I, the method comprises the steps of:

Providing Bead having a plurality of amino functional groups;

Contacting the Bead with a carboxylic acid of the formula:

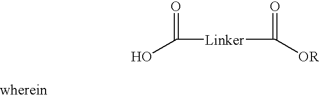

R is C₁₋₆alkyl;

Linker comprises a linear backbone having has between 2 and 12 carbon atoms and from 0 to 6 heteroatoms selected from N, O and S in the linear backbone, where each carbon of the linear backbone may be optionally substituted with 0,1, or 2 groups selected from hydrogen, C₁₋₆alkyl, hydroxy, amino, halogen, C₁₋₆alkoxy, or oxo;

under conditions conducive to the formation of a functionalized Bead according to the formula:

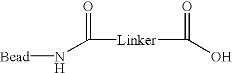

contacting the functionalized Bead with one or more equivalents of β-alanine-O-methyl ester under conditions conducive to coupling the formation of an amide bond;

contacting the polymer having at least one β-alaninyl coupled thereto with ethanolamine under conditions conducive to the formation of a solid support according to Formula I.

In another embodiment, the present invention provides a substrate for TE domain catalyzed macrocylization according to Formula V:

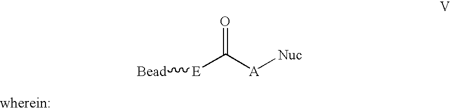

A is a peptidic sequence, synthetic hydrocarbon group, a polyketide, —(CH₂CH₂O)ₛ—, or a combination thereof having at least about 10 atoms in a linear backbone;

s is an integer from 1 to about 10;

Nuc is NH₂ or OH; and

Bead [symbol] E is a solid support having at least one amino functional group.

Preferred solid support substrates of the invention include those where A is a peptidic sequence comprising 5 to about 24 amino acid residues, or more preferably 6 to 18 amino acid residues.

Other preferred solid support bound substrates of Formula V include those substrates where the linking group comprises at least one peptidic sequence, at least one polyketide sequence, and optionally one or more groups selected from a synthetic hydrocarbon group, —(CH₂CH₂O)ₛ—, or a combination thereof such that A has at least about 10 atoms in a linear backbone.

Yet other preferred solid support substrate of the invention include those where Bead [symbol] E is a solid support according to any one of Formula I-IV.

Particularly preferred solid support bound substrates of the invention include peptidic substrates according to Formula VI:

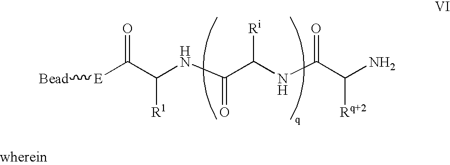

Rⁱ are independently selected for each value of i from 1 to q+2 from the group consisting of hydrogen, synthetic and biosynthetic amino acid residue side chains and side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where

each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol;

q is an integer from about 3 to about 22; and

Bead [symbol] E is a solid support having at least one amino functional group.

Preferred substrates according to Formula V or VI include substrates where E is O, and substrates where Bead is a poly(ethylene glycol acrylamide).

Preferred substrates according to Formula V and VI include solid support bound substrates according to Formula VII:

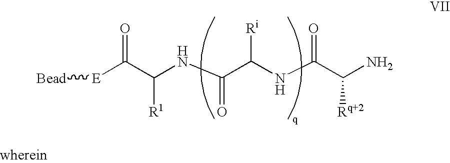

Rⁱ are independently selected for each value of i from 1 to q+2 from the group consisting of hydrogen, synthetic and biosynthetic amino acid residue side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where

each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol;

the amino acid residue bearing the R^{q+2} side chain had stereochemistry as shown in Formula VII;

q is an integer from about 3 to about 22; and

Beadz,900 E is a solid support comprising at least one amino functional group.

In another embodiment, the invention provides solid support bound substrates according to Formula V where

Nuc is OH; and

A is a polyketide or a hybrid polyketide-synthetic hydrocarbon group having from about 10 to 40 atoms in a linear.

Preferred solid support bound substrates having a polyketide or hybrid polyketide-synthetic hydrocarbon group include substrates according to Formula VIII:

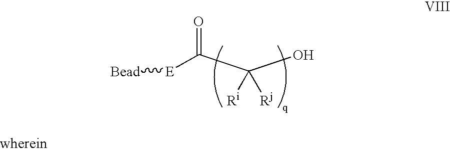

Rⁱ is independently selected for each value of i from 1 to q from the group consisting of hydrogen, C₁₋₆alkyl, hydroxy, halogen, C₁₋₆alkoxy;

R^{j} is independently selected for each value of j from 1 to q from the group consisting of hydrogen, C₁₋₆alkyl; or

CRⁱR^{j}, taken in combination, is a keto group;

where carbon having inequivalent Rⁱ and R^{j} groups for each i between 1 and q inclusive may be a (R), (S) or racemic stereocenter; and

q is an integer from about 10 to about 24.

In yet another embodiment, the invention provides solid support bound substrates for TE domain catalyzed macrocylization according to Formula V:

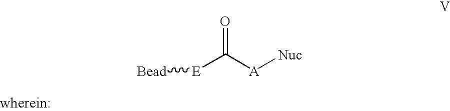

A comprises at least one peptidic sequence, at least one polyketide sequence, and optionally one or more groups selected from a synthetic hydrocarbon group, —(CH₂CH₂O)ₛ—, or a combination thereof such that A has at least about 10 atoms in a linear backbone;

s is an integer from 1 to about 10;

Nuc is NH₂ or OH; and

Bead [symbol] E is a solid support according to Formula I:

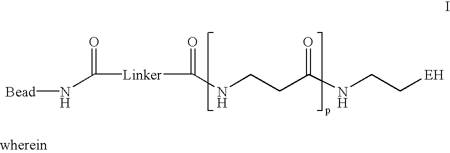

E is S or O;

p is an integer from 0-2;

Linker comprises a linear backbone having between about 4 carbon atoms and about 20 carbon atoms and between about 0 and 10 hetero atoms selected from N, O or S, where each carbon of the linear backbone may be optionally substituted with 0, 1, or 2. groups selected from C₁₋₆alkyl, hydroxy, amino, halogen, C₁₋₆alkoxy, or oxo; and

Bead is a solid particle having amino functional groups to which a linker can be attached.

Hybrid substrates of Formula V having a mixture of peptide domains and polyketide domains preferably comprise between about 4 and about 24 amino acid residues and between about 2 and about 25 ketide residues, more preferably between about 6 and 16 amino acid residues and between about 4 and about 12 ketide residues.

Preferred polyketide/peptide hybrid substrates of the invention include those substrates according to Formula IX:

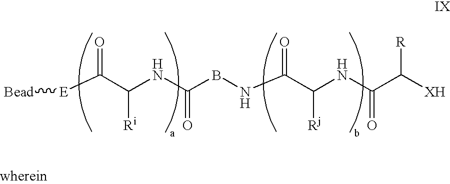

B is a polyketide residue having between about 2 and about 12 carbon atoms in a linear chain which may include one or more amide linkages or double bonds in the linear chain and which may be optionally substituted with between one and 12 groups selected from C₁₋₆alkyl, C₂₋₆alkenyl, C₂₋₆alkynyl, C₃₋₈cycloalkyl, hydroxy, or C₁₋₆alkoxy;

R, Rⁱ, and R^{j} are independently selected for each value of i from 1 to a and for each value of j from 1 to b from the group consisting of hydrogen, synthetic and biosynthetic amino acid residue side chains including side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where

each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol;

a and b are independently selected integers from about 2 to about 10;

X is O or NH; and

Bead [symbol] E is a solid support of the invention according to any one of Formula I-IV.

Particularly preferred substrates of the invention include those substrates according to Formula X:

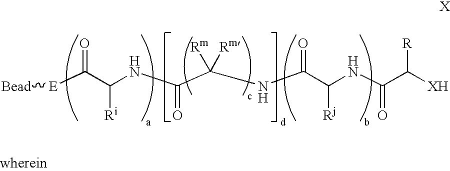

R, Rⁱ, and R^{j} are independently selected for each value of i from 1 to a and for each value of j from 1 to b from the group consisting of hydrogen, synthetic and biosynthetic amino acid residue side chains including side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where

each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol;

R^{m} and R^{m}′ are independently selected for each value of m from 1 to m from the group consisting of hydrogen, C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, and hydroxyC₁₋₆alkyl, or

R^{m} and R^{m}′ taken in combination form a keto group, or

adjacent (CR^{m}R^{m}′) residues, taken in combination, form an optionally substituted 1,2-vinylidene group;

a and b are independently selected integers from about 2 to about 10;

c is an integer of from about 2 to about 12;

d is an integer of from about 1 to about 5;

X is O or NH; and

Bead [symbol] E is a solid support of the invention according to any one of Formula I-IV.

Particularly preferred substrates of Formula IX or Formula X include those in which the group X is NH and those substrates in which the group X is O.

Other preferred substrates of Formula X, include those substrates wherein each of the d residues in Formula X according to the formula:

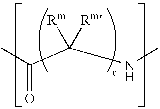

are independently selected from groups according to Formula XI:

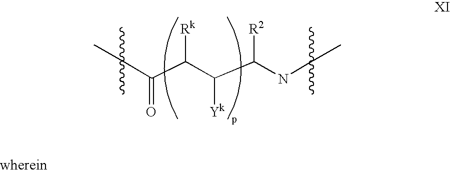

R² and each occurrence of R^{k} for each value of k from 1 to p are independently selected from the group consisting of hydrogen, C₁₋₆alkyl, C₂₋₆alkenyl, C₂₋₆alkynyl, C₃₋₈cycloalkyl, C₇₋₁₂aralkyl, hydroxy, C₁₋₆alkoxy, and hydroxyC₁₋₆alkyl;

Y is independently selected for each value of k from 1 to p from the group selected from hydrogen, hydroxy, C₁₋₆alkoxy, C₁₋₆alkyl, or keto; or

—(CHR^{k}CHY)— taken in combination form a 1,2-vinylidene group

p is an integer of from about 1 to about 5

Particularly preferred groups according to Formula XI which are suitable for use in substrates according to Formula X include groups selected from:

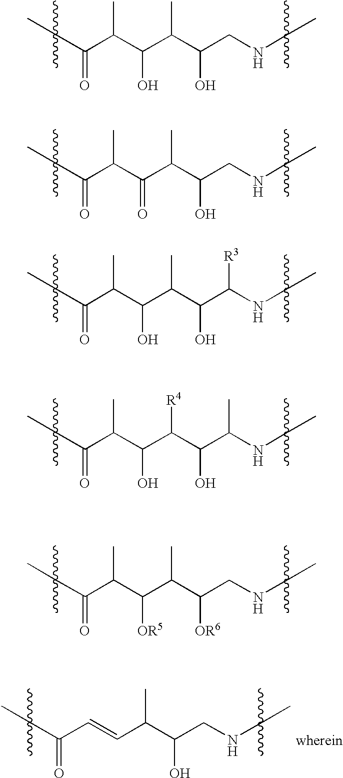

R³ is a synthetic and biosynthetic amino acid residue side chains including side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol; and

R⁴, R⁵, and R⁶ are independently selected from hydrogen and optionally substituted C₁₋₆alkyl.

The invention provides substrates in which each stereocenter of each ketide or amino acid residue may be either (R) or (S) configuration such that each substrate according to any one of Formula V and VIII-X including those groups of Formula XI may be an individual stereoisomer, a racemate or a mixture of diastereomers.

In preferred embodiments, the invention provides substrates bound to a solid support of Formula IX which are suitable for cyclization by the TE domain excised from the tyrocidine NRPS, which substrates are represented by Formula XII:

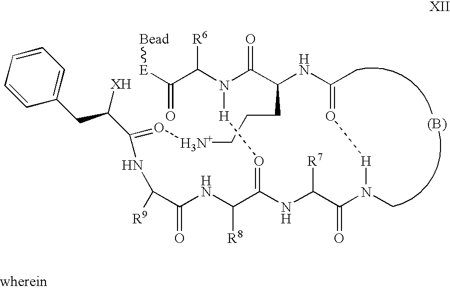

B is a polyketide residue having between about 2 and about 12 carbon atoms in a linear chain which may include one or more amide linkages or double bonds in the linear chain and which may be optionally substituted with between one and 12 groups selected from C₁₋₆alkyl, C₂₋₆alkenyl, C₂₋₆alkynyl, C₃₋₈cycloalkyl, hydroxy, or C₁₋₆alkoxy;

R⁶, R⁷, R⁸, and R⁹ are independently selected from the group consisting of synthetic and biosynthetic amino acid residue side chains including side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol; and

X is O or NH.

The invention also provides hybrid macrocyclic molecules which are prepared by TE domain catalyzed macrocyclicization of linear substrates according to any one of Formula V, IX, and X. Preferred hybrid macrocycles having peptide and polyketide domains include macrocycles according to Formula XIII:

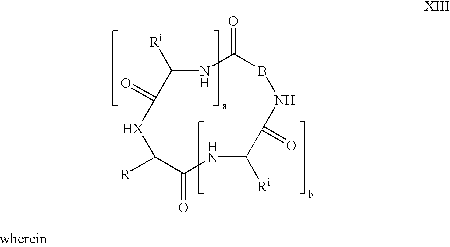

B is a polyketide residue having between about 2 and about 12 carbon atoms in a linear chain which may include one or more amide linkages or double bonds in the linear chain and which may be optionally substituted with between one and 12 groups selected from C₁₋₆alkyl, C₂₋₆alkenyl, C₂₋₆alkynyl, C₃₋₈cycloalkyl, hydroxy, or C₁₋₆alkoxy;

R, Rⁱ, and R^{j} are independently selected for each value of i from 1 to a and for each value of j from 1 to b from the group consisting of hydrogen, synthetic and biosynthetic amino acid residue side chains including side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where

each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol;

a and b are independently selected integers from about 2 to about 10; and

X is O or NH.

Particularly preferred macrocyclic compound of Formula XIII provided by the invention include macrocycles according to Formula XIV:

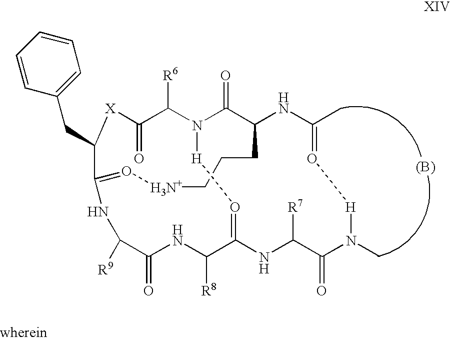

B is a polyketide residue having between about 2 and about 12 carbon atoms in a linear chain which may include one or more amide linkages or double bonds in the linear chain and which may be optionally substituted with between one and 12 groups selected from C₁₋₆alkyl, C₂₋₆alkenyl, C₂₋₆alkynyl, C₃₋₈cycloalkyl, hydroxy, or C₁₋₆alkoxy;

R⁶, R⁷, R⁸, and R⁹ are independently selected from the group consisting of synthetic and biosynthetic amino acid residue side chains including side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol; and

X is O or NH.

The present invention provides methods of preparing a linear peptidic sequence bound to a solid support of the invention, the method comprising the steps of:

providing a solid support according to Formula I which comprises a Bead having a plurality of amino groups and at least one Linker coupled to the polymer through an amino group;

contacting the solid support having a Linker with a series of amino acid residues under conditions conducive to the formation of a specified amino acid sequence coupled to the EH group of the Linker to form a linear peptidic sequence bound to a solid support.

More specifically the methods for the preparation of a linear peptidic sequence bound to a solid support comprise the steps of:

(a) providing a solid support according to any one of claims 1 through 9 which comprises a solid particle having a plurality of amino groups and at least one Linker coupled to the solid particle through at one of the amino groups;

(b) contacting a N-protected amino acid residue with the solid support under conditions conducive to coupling the free-carboxylate of the amino acid with at least a portion of the EH residues of the polymer support to form an ester or thioester bond;

(c) contacting the solid support having a N-protected amino acid coupled thereto with a combination of chemicals suitable for the deprotection of the N-protected amino-acid group;

(d) contacting a N-protected amino acid residue with the solid support having an amino acid sequence coupled to the Linker under conditions conducive to formation of an amide bond to extend the amino acid sequence by one residue;

(e) contacting the polymer solid support having an amino acid sequence coupled thereto with a combination of chemicals suitable for the deprotection of the protected N-terminal amino group; and

(f) repeating steps (d) and (e) to synthesize a specified amino acid coupled to the EH group of the Linker to form a linear peptidic sequence bound to a polymer support.

The invention also provides methods of preparing a linear sequence having peptide and polyketide domains bound to a solid support of the invention, the method comprising the steps of:

providing a solid support according to Formula I which comprises a Bead having a plurality of amino groups and at least one Linker coupled to the polymer through an amino group;

contacting the solid support having a Linker with a series of amino acid residues and polyketide residues under conditions conducive to the formation of a specified amino acid and ketide sequence coupled to the EH group of the Linker to form a linear hybrid substrate having peptide domains and polyketide domains bound to a solid support.

Preferred methods for the preparation of a linear peptidic sequence bound to a solid support include the use of poly(ethylene glycol acrylamide) as the Bead. Additional preferred methods include the use of solid supports of Formula I where E is O. Most preferred methods of preparing a linear peptidic sequence bound to a solid support include the use of poly(ethylene glycol acrylamide) as the Bead, where E is oxygen such that the amino acid sequence is prepared using standard fluorenyl methyl oxycarbonyl (FMOC) amino acid coupling techniques. Typically a solid support bound substrate synthesized by the methods of the invention comprises between about 5 and about 24 amino acid residues.

Other preferred methods for the preparation of a linear peptidic sequence or a linear hybrid substrate having polyketide and amino acid sequences bound to a solid support include the use of poly(ethylene glycol acrylamide) as the Bead. Additional preferred methods include the use of solid supports of Formula A where E is O. Most preferred methods of preparing a linear peptidic sequence or a linear hybrid substrate having polyketide and amino acid sequences bound to a solid support include the use of poly(ethylene glycol acrylamide) as the Bead, where E is oxygen such that the amino acid sequence or hybrid polyketide/amino acid sequence is prepared using standard fluorenyl methyl oxycarbonyl (FMOC) amino acid coupling techniques. Typically a solid support bound substrate synthesized by the methods of the invention comprises between about 5 and about 24 amino acid residues and optionally between about 2 and 25 ketide residues.

In yet another embodiment, the present invention provides methods of formation of macrocyclic molecules by TE domain catalyzed cyclization of solid support bound substrates, the method comprising:

providing a substrate according to Formula V which comprises an activated acyl residue and a pendant nucleophile separated by a linear backbone where the activated acyl residue is coupled to a solid support:

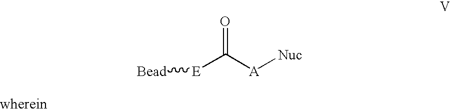

A is a peptidic sequence, synthetic hydrocarbon group, a polyketide, —(CH₂CH₂O)ₛ—, or a combination thereof having at least about 10 atoms in a linear backbone;

s is an integer from 1 to about 10;

Nuc is NH₂ or OH; and

Bead [symbol] E is a solid particle comprising at least one amino functional group; and

contacting a purified TE domain protein with the solid support bound substrate under conditions conducive to formation of an transient TE-O-acyl bond and subsequent formation of a macrocyclic product by displacement of a TE domain by a pendant nucleophile.

In preferred methods of forming macrocycles provided by the invention, the solid support bound substrate comprises a peptidic A group which comprises between about 5 to about 24 amino acid residues, or more preferably 6 to about 18 amino acid residues.

In other preferred methods of forming macrocycles provided by the invention, the group A comprises at least one peptidic sequence, at least one polyketide sequence, and optionally one or more groups selected from a synthetic hydrocarbon group, —(CH₂CH₂O)ₛ—, or a combination thereof such that A has at least about 10 atoms in a linear backbone

In yet other preferred methods of forming macrocycles provided by the invention, the solid support bound substrate comprises a peptidic A group which comprises between about 5 to about 24 amino acid residues, or more preferably 6 to about 18 amino acid residues. Hybrid substrates of Formula I having a mixture of peptide domains and polyketide domains preferably comprise between about 4 and about 24 amino acid. residues and between about 2 and about 25 ketide residues, more preferably between about 6 and 16 amino acid residues and between about 4 and about 12 ketide residues.

Particularly preferred solid support bound substrates for macrocyclization methods of the invention catalyzed by an excised TE domain include solid support bound substrates according to Formula V-XII having peptidic, polyketide or a hybrid polyketide-synthetic hydrocarbon group as the A group linear backbone of the substrate according to Formula V.

In another embodiment, the present invention features a method of forming a library of macrocyclic molecules from a library of solid support bound substrates, the method comprising the steps of:

providing a plurality of solid support bound substrates suitable for macrocyclization according to Formula V:

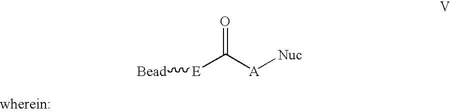

A is a peptidic sequence, synthetic hydrocarbon group, a polyketide, —(CH₂CH₂O)ₛ—, or a combination thereof having at least about 10 atoms in a linear backbone;

s is an integer from 1 to about 10;

Nuc is NH₂ or OH;

Bead [symbol] E is a solid support having at least one amino functional group;

where each solid support substrate of the library of solid support bound substrates comprises a chemically distinct combination of A and Nuc; and

contacting purified excised TE domain protein with each solid support bound substrate having a chemically distinct combination of A and Nuc under conditions conducive to formation of an transient TE-O-acyl bond and subsequent formation of a macrocyclic product by displacement of a TE domain by a pendant nucleophile, Nuc such that a plurality of chemically distinct macrocycles are formed.

Preferred libraries including those prepared from a plurality of solid support bound substrates of Formula V wherein the group A comprises at least one peptidic sequence, at least one polyketide sequence, and optionally one or more groups selected from a synthetic hydrocarbon group, —(CH₂CH₂O)ₛ—, or a combination thereof such that A has at least about 10 atoms in a linear backbone.

In preferred methods of library formation, each solid support bound substrate having a distinct A and Nuc combination is physically segregated from other solid support bound substrates of the library of solid support bound substrates such that chemically distinct macrocycles of the library are segregated after excised TE domain catalyzed macrocyclization. Typically each solid support bound substrate charged into a different well of one or more multi-well plates.

Preferred libraries of solid support bound substrates of the invention include substrates having an A group which is peptidic sequence which is chemically distinct for each solid support bound substrate of the library, where each peptidic sequence typically comprises 5 to about 24 or preferably 6 to 18 amino acid residues and diversity in the A group of each substrate is generated by varying one, two or more residues of the A group peptidic sequence while keeping the other amino acid residues of the peptidic sequence substantially the same across the A groups of all the solid support bound substrates of the library.

Other preferred libraries of solid support bound substrates of the invention include substrates having an A group which is a peptide sequence or a hybrid peptide/polyketide sequence which is chemically distinct for each solid support bound substrate of the library, where each peptide or hybrid peptide/polyketide sequence typically comprises 5 to about 24 or preferably 6 to 18 amino acid residues, between about 2 and about 25 ketide residues, more preferably between about 4 and about 12 ketide residues. Preferably diversity in the A group of each substrate is generated by varying one, two or more amino acid residues or one or more substitutents of the polyketide sequence of the A group peptidic sequence or hybrid peptide/polyketide sequence while keeping the other amino acid residues and/or polyketide residues of the substrate substantially the same across the A groups of all the solid support bound substrates of the library.

In another embodiment of the invention, the solid supports of the invention according to formula I or any subformula thereof are suitable for use in assays. Typically a library of solid support bound substrates is screened against an enzyme to determine the substrate specificity of the enzyme. In preferred embodiments libraries of solid support bound substrates according to any of Formula V-XII or any subformulae thereof are suitable for use in screening which substrates are suitable for macrocyclization using a specified TE domains.

In another embodiment, a library of solid support bound substrates according to Formula IX where each substrate has a different fatty acid hydrophobic group R is suitable for screening substrate specificity of fatty acid synthase in hydrolyzing fatty acid substrates bound to solid supports of the invention.

Preferred solid support bound fatty acid substrates include those according to Formula XV:

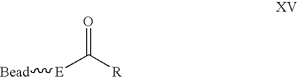

wherein:

R is a fatty acid hydrophobic residue having between about 4 and about 36 carbon atoms; and

Bead [symbol] E is a solid support having at least one amino functional group.

In accord with the present invention, the preparation of macrocyclic molecules comprises contacting purified excised TE domain protein with a substrate molecule bound to a solid support through a linker that is to be cyclized. The solid support bound substrate molecule, such as those of Formula V-XII, typically comprises an activated acyl residue and a pendant nucleophile separated by a linear backbone. The excised TE domain protein and substrate are contacted under conditions conducive to formation of a TE-O-acyl bond such that subsequently the pendant intramolecular nucleophile can displace the TE domain to form the macrocyclic product. Examples of suitable substrate molecules for macrocyclization catalyzed by the excised TE domain from tyrocidine synthetase are included in compounds represented by Formula V, particularly those substrates represented by Formula VI and VII.

Other substrate molecules are suitable for macrocyclization by excised TE domain proteins originating from other NRPS or PKS multidomain systems. Specific examples of the invention describe the use of TE domain protein excised from the Tyrocidine A NRPS multidomain enzyme and or from the surfactin synthetase multidomain to catalyze macrocyclization of substrate molecules. However, the use of other excised TE domain proteins that can be used to catalyze the macrocyclization of other substrates. An appropriate excised TE domain protein can be chosen to catalyze a specified substrate based on structure commonalties between the specified substrate and the wild-type substrate of a particular TE domain protein. For example, excised TE domain proteins from PKS multidomain enzymes are preferable catalysts for the macrocyclization of polyketide substrates and excised TE domain proteins from NRPS multidomain enzymes are preferable for polypeptide substrates or substrates that comprise one or more peptide sequences. Suitable excised TE domain proteins for use in the present invention include, but are not limited to tyrocidine synthetase TE, gramicidin synthetase TE, surfactin synthetase TE, bacitracin synthetase TE, fengycin synthetase TE, calcium-dependent antibiotic (CDA) synthetase TE, microcystin synthetase TE, epothilone synthetase TE, daptomycin synthetase TE, syringomycin synthetase TE, nystatin synthetase TE, lichenysin synthetase TE, 6-deoxyerythronolide B synthase (DEBS), actinomycin synthetase TE, pristinamycin synthetase TE, virginiamycin synthetase TE, picromycin synthetase TE, and the like.

In preferred embodiments, TE domain protein catalyzed macrocyclization reactions are carried out in an aqueous medium. The aqueous medium also can comprise buffers such as 3-(N-morpholino)propanesulfonic acid (MOPS) and the like so that the aqueous solution has a pH between about 6 and about 9. Preferably, the pH is between about 6.5 and about 8. Particularly preferred are methods wherein the macrocyclization is carried out in about pH 7 aqueous medium.

Organic co-solvents are tolerated by the macrocyclization method where the organic solvent or a solution of two or more organic solvents is less than about 20% v/v of the solution. Preferably, the organic solution is less than about 10%, 5%, 2% or 1% v/v of the aqueous solution. Preferred organic solvent additives or organic co-solvents, if utilized, are miscible with water at the % v/v of the aqueous solution and are poor nucleophiles so that the organic solvent generally does not compete with the intramolecular nucleophile at displacing the TE-O-acyl bond. Preferable organic co-solvents are dimethylsulfoxide (DMSO), N,N-dimethyl-formamide (DMF) and other polar, weakly nucleophilic organic liquids.

Macrocyclization reactions are preferably carried out in a medium that is capable of inducing sufficient level of swelling in the polymer of the solid support such that substrates may be synthesized onto the polymer bound Linker groups and TE domains may diffuse though the polymer matrix. Additionally preferred reaction mediums for macrocyclization reactions of the invention are capable of solvating the macrocyclic molecule generated in the cyclization reaction. Typical media include water, buffered water solutions, water/detergent mixtures, and water/organic solutions comprising a substantial portion of water. Preferably, the solubility of the macrocyclic molecule product in the reaction mixture is at least about 0.1 g/L. More preferably, the solubility of the macrocyclic molecule product in the reaction mixture is at least about 1 g/L.

Preferred aqueous detergent mixtures typically comprise between 0.01% to about 5% by weight detergent in water, more preferably between about 0.05% to about 1% by weight detergent in water or about 0.1% by weight detergent in water. Although any common detergent is suitable for use in reaction media of the present invention, aqueous solutions of BRIJ-58 or Triton X-100 are particularly preferred because these solutions result in higher yield for macrocyclizations susceptible to hydrolysis.

The quantity of catalyst used depends upon the rate of catalysis for a particular substrate, the volume of solution and other environmental factors. Typical catalyst loadings are less than about 20 mole % based on the moles of substrate. Preferred catalyst loadings are less than about 10 mole %, more preferably less than about 5 mole %. Particularly preferred ranges of catalyst loadingare about 0.1 to about 2 mole %, more preferably from about 0.1 to about 1 mole %.

Preferably, macrocyclization reactions in accord with the present invention are performed at between about 0° C. and about 40° C., more preferably between about 10° C. and about 30° C. In particularly preferred macrocyclization reactions in accord with the present invention are performed at about room temperature, i.e., 20-25° C. However, the temperature can be varied as long as the TE domain protein is sufficiently stable and active.

Macrocyclization reactions of the present invention typically are complete in about 5 seconds to about 24 hours. Preferably, macrocyclization reactions are complete in less than about 1 hour. More preferably, macrocyclization reactions are complete in less than about 5 minutes. Macrocyclization reactions of the invention that are conducted in a detergent/water media frequently take longer than other macrocyclization reactions of the invention. Typical reaction times for macrocyclization in a detergent/water media range from 0.5 hours to about 24 hours or more preferably between about 1 hour and about 16 hours.

Macrocyclization substrates are preferably cyclized by the excised TE domain protein having a rate constant (k_{cat}) that is at least about 1 cyclization reaction per minute per enzyme molecule. Macrocyclization substrates are more preferably cyclized by the excised TE domain protein having a rate constant (k_{cat}) that is at least about 10 cyclization reactions per minute per enzyme molecule.

The term K_{M} is defined as the concentration at which the observed rate of cyclization is equal to one-half the maximum observed rate of cyclization. Macrocyclization substrates are preferably cyclized by the excised TE domain protein at a rate equal to one-half the maximum rate at a concentration of less than 1 mM (i.e., K_{M}<1 mM). Macrocyclization substrates are more preferably cyclized by the excised TE domain protein at a rate equal to one-half the maximum rate at a concentration of less than 0.1 mM (i.e. K_{M}<0.1 mM).

Preferred non-peptidic spacers of the present invention comprise one or a combination of more than one of the following optionally substituted groups that include C₁-C₁₂-alkyl, C₂-C₁₂-alkenyl, C₂-C₁₂-alkynyl, C₃-C₇-cycloalkyl, C₃-C₇-heteroalicyclic, aryl, heteroaryl, amine (NH), C₁-C₁₂-alkylamino, amide, ester, ketone, sulfoxide, ether, thioether, imine, sulfone, and the like. More preferable are spacers that comprise one or a combination of more than one of the following optionally substituted groups that include α,ω-alkandiyl, α,ω-alkane diol, α,ω-alkane diamine, ω-(1-alkanol)amine, ω-hydroxyalkanoate or ω-aminoalkanoate functional groups linked together by independently chosen ether, amine, amide or ester bonds.

Particularly preferred non-peptidic spacers of the present invention include one or a combination of more than one of the following optionally substituted groups glycine, glycolate, O-(2-aminoethyl)glycolate, O-(2-ethanol)glycolate, O-(2-(2-aminoethoxy)ethyl)glycolate, O-(diethylene glycol)glycolate, and the like that are linked together by either amide or ester bonds.

In other preferred embodiments of the present invention, other macrocyclization substrates, which may not meet the requirements of Formulae of the invention, can be cyclized by excised TE domain proteins other than the excised TE domain protein from tyrocidine synthetase are also suitable substrates of the invention.

Libraries of solid support bound substrates prepared by varying one or more residues of wild-type substrate may be screened against one or more TE domains to determine (1) superior TE domains for cyclization of a target substrate or to determine the tolerance of a given TE domain substrate variation at one or more points in the linear sequence of the substrate backbone, which may comprise a peptidic sequence, a polyketide sequence, a synthetic hydrocarbon sequence or a combination thereof.

In additional preferred embodiments of the invention, macrocyclic molecules prepared by the methods of the present invention can have useful pharmaceutical applications that include but are not limited to use as antibiotics, antitumor agents, cholesterol-lowering drugs, and immunosuppressants. Other applications and molecules with other biological activity profiles are also suitable for the present invention.

The solid supports, solid support bound substrates and methods of using solid support bound substrates of the invention provided by the present invention are suitable for use in the production of very large and complex libraries of pure macrocyclic molecules from simple amino acid starting materials. In particularly preferred embodiments where solid supports according to formula I have a hydroxy terminus, e.g., E is O, the methods of synthesizing solid support bound peptidic substrates are amenable to standardized FMOC-protected peptide techniques that are amenable to robotic automation. Macrocycles prepared using the methods of the invention may be obtained in high yields and high regioselectivities without the use of organic solvents or extensive protecting group strategies. Additionally macrocycles prepared by the methods of the invention may have useful pharmaceutical applications as antibiotics, antitumor agent, cholesterol-lowering drugs, immunosuppressants, synthetic hormones, pesticides or the like.

The methods of the invention for macrocycle synthesis are be particularly useful in the parallel or combinatorial synthesis of large libraries of macrocyclic compounds which may be screened for useful biological activity. Because many known bioactive molecules are macrocycles, preparation of a diverse library of macrocyclic compounds would be an attractive strategy for drug discovery.

All publications disclosed herein are incorporated herein by reference. The following non-limiting examples are illustrative further of the invention.

### EXAMPLE 1

### Synthesis of Pantebead Resin and Subsequent Solid Phase Peptide Synthesis

Synthesis of the Pantebead resin begins with polyethylene glycol acrylamide (PEGA) resin (Renil M, Meldal M, et al., J. Peptide Sci., 1998, 4, 195-210) terminating in a free amine moiety. Solid phase peptide coupling of monomethyl suberic acid to the resin was performed by preincubating the acid (5 eq) with HBTU (O-benzotriazol-1-yl-N,N,N′,N′-tetramethyluronium hexafluorophosphate) (4.9 eq), HOBt (1-hydroxybenzotriazole hydrate) (5 eq.), and DIEA (diisopropylethylamine) (10 eq.) in DMF for 10 minutes followed by addition to the resin and agitation for 2 hours. The resin was washed 5× with DMF. The above coupling step was repeated a second time with agitation overnight.

The terminal methyl ester was deprotected to the free acid with THF/MeOH/10N NaOH (3/1.5/0.5) and agitation for 30 minutes, followed by acidification by MeOH/2N HCl (5/1) followed by a wash 2× with water and 2× with MeOH. This deprotection step was repeated a second time, and the resin was washed 2× with MeOH, 2× with water, 2× with MeOH, and 3× with DMF. This yields substance 1A.

Coupling of beta-alanine methyl ester hydrochloride was carried out with preincubation of the resin with HBTU (4.9 eq), HOBt (5 eq), and DIEA (10 eq) in DMF for 10 minutes followed by addition of beta-alanine methyl ester hydrochloride (5 eq.) and agitation for 2 hours. The resin was washed 3× with DMF and the coupling step repeated exactly a second time.

Deprotection of the terminal methyl ester was performed exactly as the previous methyl ester deprotection. This yields substance 2A. Coupling of ethanolamine was carried out with preincubation of the resin with HBTU (4.9 eq), HOBt (5 eq), and DIEA (10 eq) in DMF for 10 minutes followed by addition of ethanolamine hydrochloride (20 eq.) and agitation for 2 hours. The resin was washed 3× with DMF, 2× with MeOH, and 3× with DMF. A second coupling was performed with a different coupling reagent. Ethanolamine hydrochloride (20 eq), PyBOP (benzotriazole-1-yl-oxy-tris-pyrrolidino-phosphonium hexafluorophosphate) (4.9 eq), HOBt (5 eq), and DIEA (10 eq) were all added to the resin in DMF and agitated overnight. The resin was then washed 2× with DMF, 2× with dichloromethane, 2× with MeOH, 2× with water, 2× with MeOH, 2× with dichloromethane, 2× with DMF. This gives the free Pantebeads, 3A.

### EXAMPLE 2

### Synthesis of Peptides on Pantebeads

We carried out the synthesis of large amounts peptide-linked Pantebeads on a continuous-flow solid phase peptide synthesizer with double-couplings at each step for fidelity. For synthesis of libraries, the beads were distributed into 96-well polyethylene filter plates (Whatman), and reagent addition was performed manually followed by clamping the filter plate in a sealed clamp (Whatman Combi-Clamp) and agitation on a circular agitator. The plates were evacuated via a filter plate vacuum manifold and washed with solvent addition from above. All couplings, piperidine deprotections, and final TFA deprotection was carried out in the filter plates.

Synthesis on Pantebeads follows standard FMOC peptide synthesis techniques with DIPCDI and HOBt coupling of FMOC protected amino acids and HOBt coupling with pentafluorophenol ester (PFP) activated/FMOC-protected amino acids. (W. C. Chan and P. D. White, “Fmoc Solid Phase Peptide Synthesis: A Practical Approach”, Oxford University Press, Oxford, 2000.) Attachment of the first amino acid to the hydroxy-terminal Pantebead resin was carried out by standard technique, with MSNT (1-(mesitylene-2sulphonyl)-3-nitro-1H-1,2,4-triazole) and methylimidazole. FMOC peptide coupling of additional amino acids follows. Final deprotection was carried out by traditional methods with TFA and triisopropylsilane and water followed by wash 3× with DMF and 3× with buffered water (MOPS pH 7.0).

Peptide libraries may also be carried out as libraries of complex mixtures (utilizing split-and-pool combinatorial methodology) or libraries of organized mixtures (iterative, positional scanning, or orthogonal libraries). (Lebl M, and Krchnak V, Methods Enzym., 1997, 289, 337-392.) D- and L-amino acids, non-natural amino acids, and peptoids may all be incorporated into the library composition for molecular diversity.

Although the invention has been described including preferred embodiments thereof and using specific terms, such description is for illustrative purposes only, and it is to be understood that changes and variations may be made by those skilled in the art without departing from the spirit or scope of the invention as set forth in the following claims.

What is claimed is:

1. A solid support suitable for solid phase synthesis according to Formula I: 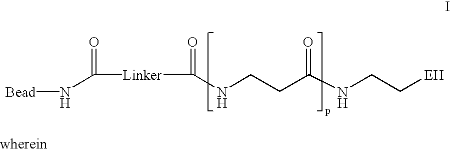
E is S or O;
p is an integer from 0-2;
Linker comprises a linear backbone having between about 4 carbon atoms and about 20 carbon atoms and between about 0 and 10 hetero atoms selected from N, O or S, where each carbon of the linear backbone may be optionally substituted with 0, 1, or 2 groups selected from C₁₋₆alkyl, hydroxy, amino, halogen, C₁₋₆alkoxy, or oxo; and
Bead is a solid particle having amino functional groups to which a linker can be attached.

2. A solid support of claim 1, according to Formula II: 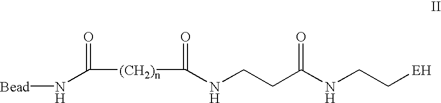
E is O or S;
n is an integer from about 3 to about 12; and
Bead is a solid particle having amino functional groups to which a linker can be attached.

3. A solid support of claim 1, according to the Formula III: 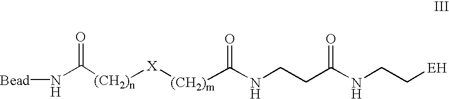
n and m are independently selected integers from about 1 to about 10
m+n is about 3 to about 12;
E is O or S;
X is —CH(OH)—, —CH(CH₃)—, —C(CH₃)₂—, —S—, —O—, —S(O)—, or —S(O)₂—; and
Bead is a solid particle having amino functional groups to which a linker can be attached.

4. A solid support of claim 4, wherein
m and n are independently selected integers from about 1 to about 6;
m+n is from about 4 to about 8;
E is O; and
X is —S(O)— or —S(O)₂—.

5. A solid support according to Formula IV: 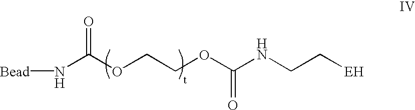
wherein
E is O or S;
t is an integer from about 1 to about 9; and
Bead is a solid particle having amino functional groups to which a linker can be attached.

6. A solid support of claim 1 wherein E is O.

7. A method of preparation of a solid support of Formula I, comprising:
providing a solid particle having a plurality of amino functional groups;
contacting the bead with a carboxylic acid of the formula: 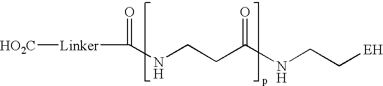
E is S or O;
p is an integer from 0-2; and
Linker comprises a linear backbone comprising between about 4 carbon atoms and about 20 carbon atoms and between about 0 and 10 hetero atoms selected from N, O or S, where each carbon of the linear backbone may be optionally substituted with 0, 1, or 2 groups selected from C₁₋₆alkyl, hydroxy, amino, halogen, C₁₋₆alkoxy, or oxo;
under conditions conducive to the formation of a solid support according to Formula I:

8. A method of preparation of a solid support of Formula I where p is 1, comprising:
providing an insoluble polymer having a plurality of amino functional groups;
contacting the polymer with a carboxylic acid of the formula: 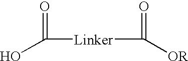
E is S or O;
R is C₁₋₆alkyl;
Linker comprises a linear backbone comprising between 2 and 12 carbon atoms and from 0 to 6 heteroatoms selected from N, O and S in the linear backbone, where each carbon of the linear backbone may be optionally substituted with 0,1, or 2 groups selected from hydrogen, C₁₋₆alkyl, hydroxy, amino, halogen, C₁₋₆alkoxy, or oxo;
under conditions conducive to the formation of a functionalized solid support according to the formula: 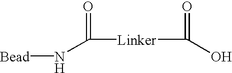
contacting the functionalized solid support with β-alanine-O-methyl ester under conditions conducive to coupling the formation of an amide bond;
contacting the polymer having a β-alaninyl coupled thereto with ethanolamine under conditions conducive to the formation of a solid support according to Formula II.

9. A substrate for TE domain catalyzed macrocylization according to Formula V: 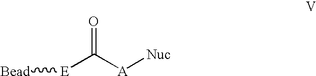
wherein:
A is a peptidic sequence, synthetic hydrocarbon group, a polyketide, —(CH₂CH₂O)ₛ—, or a combination thereof having at least about 10 atoms in a linear backbone;
s is an integer from 1 to about 10;
Nuc is NH₂ or OH; and
Bead [symbol] E is a solid support according to claim 1.

10. A substrate of claim 9, according to Formula VI: 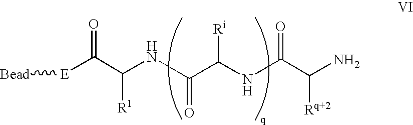
Rⁱ are independently selected for each value of i from 1 to q+2 from the group consisting of hydrogen, synthetic and biosynthetic amino acid residue side chains including side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where
each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol;
q is an integer from about 3 to about 22; and
Bead [symbol] E is a solid support according to any one of claims 1 to 9.

11. A substrate of claim 10 according to the Formula VII: 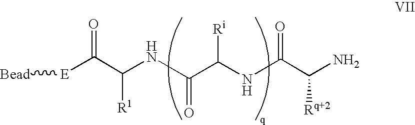
wherein
Rⁱ are independently selected for each value of i from 1 to q+2 from the group consisting of hydrogen, synthetic and biosynthetic amino acid residue side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where
each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol;
the amino acid residue bearing the R^{q+2} side chain had stereochemistry as shown in Formula VII;
q is an integer from about 3 to about 22; and
Bead [symbol] E is a solid support according to any one of claims 1 to 9.

12. A substrate of claim 11 wherein:
Nuc is OH; and
Linker is a polyketide or a hybrid polyketide-synthetic hydrocarbon group having from about 10 to 40 atoms in a linear backbone connecting the activated acyl, —C(O)E˜˜Bead group, and the Nuc group;

13. A substrate according to claim 12 according to Formula VIII: 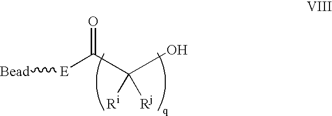
wherein
Rⁱ is independently selected for each value of i from 1 to q from the group consisting of hydrogen, C₁₋₆alkyl, hydroxy, halogen, C₁₋₆alkoxy;
R^{j} is independently selected for each value of j from 1 to q from the group consisting of hydrogen, C₁₋₆alkyl; or
CRⁱR^{j}, taken in combination, is a keto group;
where carbon having inequivalent Rⁱ and R^{j} groups for each i between 1 and q inclusive may be a (R), (S) or racemic stereocenter; and
q is an integer from about 10 to about 24.

14. A substrate according to claim 9 wherein A comprises at least one peptidic sequence, at least one polyketide sequence, and optionally one or more groups selected from a synthetic hydrocarbon group, —(CH₂CH₂O)ₛ—, or a combination thereof such that A has at least about 10 atoms in a linear backbone.

15. A substrate of claim 9, according to Formula IX: 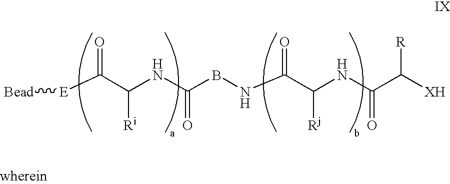
B is a polyketide residue having between about 2 and about 12 carbon atoms in a linear chain which may include one or more amide linkages or double bonds in the linear chain and which may be optionally substituted with between one and 12 groups selected from C₁₋₆alkyl, C₂₋₆alkenyl, C₂₋₆alkynyl, C₃₋₈cycloalkyl, hydroxy, or C₁₋₆alkoxy;
R, Rⁱ, and R^{j} are independently selected for each value of i from 1 to a and for each value of j from 1 to b from the group consisting of hydrogen, synthetic and biosynthetic amino acid residue side chains including side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where
each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol;
a and b are independently selected integers from about 2 to about 10;
X is O or NH; and
Bead [symbol] E is a solid support as defined in claim 9.

16. A substrate of claim 15 according to the Formula X: 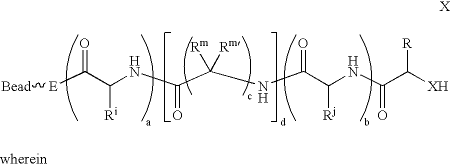
R, Rⁱ, and R^{j} are independently selected for each value of i from 1 to a and for each value of j from 1 to b from the group consisting of hydrogen, synthetic and biosynthetic amino acid residue side chains including side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where
each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol;
R^{m} and R^{m}′ are independently selected for each value of m from 1 to m from the group consisting of hydrogen, C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, and hydroxyC₁₋₆alkyl, or
R^{m} and R^{m}′ taken in combination form a keto group, or
adjacent (CR^{m}R^{m}′) residues, taken in combination, form an optionally substituted 1,2-vinylidene group;
a and b are independently selected integers from about 2 to about 10;
c is an integer of from about 2 to about 12;
d is an integer of from about 1 to about 5;
X is O or NH; and
Bead [symbol] E is a solid support as defined in claim 9.

17. A substrate of claim 16, wherein X is NH.

18. A substrate of claim 16, wherein X is O.

19. A substrate of claim 16, wherein each of the d residues in Formula X according to the formula: 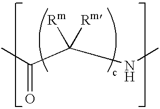
are independently selected from groups according to Formula XI: 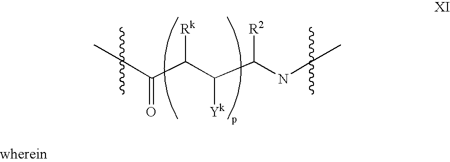
R² and each occurrence of R^{k} for each value of k from 1 to p are independently selected from the group consisting of hydrogen, C₁₋₆alkyl, C₂₋₆alkenyl, C₂₋₆alkynyl, C₃₋₈cycloalkyl, C₇₋₁₂aralkyl, hydroxy, C₁₋₆alkoxy, and hydroxyC₁₋₆alkyl;
Y is independently selected for each value of k from 1 to p from the group selected from hydrogen, hydroxy, C₁₋₆alkoxy, C₁₋₆alkyl, or keto; or
—(CHR^{k}CHY)— taken in combination form a 1,2-vinylidene group; and
p is an integer of from about 1 to about 5.

20. A substrate according to claim 19, wherein each group according to Formula XI are independently selected from the group consisting of: 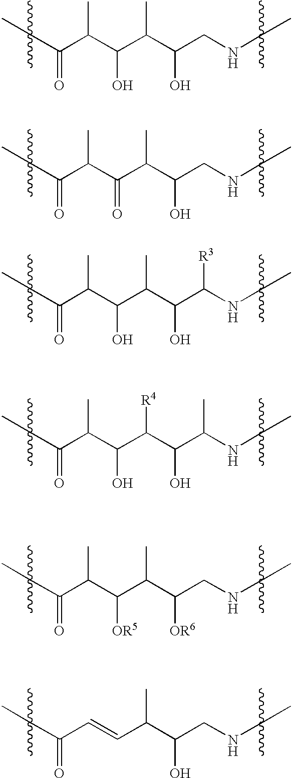
wherein
R³ is a synthetic and biosynthetic amino acid residue side chains including side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol; and
R⁴, R⁵, and R⁶ are independently selected from hydrogen and optionally substituted C₁₋₆alkyl.

21. A substrate according to claim 20, wherein each group according to Formula IV may be an individual stereoisomer, a racemate or a mixture of diastereomers.

22. A substrate of claim 14 according to Formula XII: 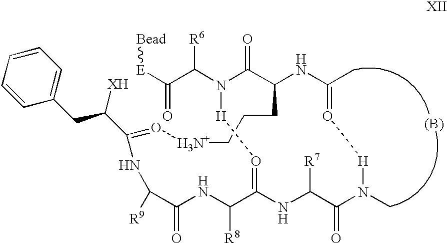
wherein R⁶, R⁷, R⁸, and R⁹ are independently selected from the group consisting of synthetic amino acid residue side chains, biosynthetic amino acid residue side chains, and side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol; and
X is OH or NH₂.

23. A method of preparation of a linear peptidic sequence bound to a solid support, the method comprising the steps of:
providing a solid support according to claim 1 which comprises a having a plurality of amino groups and a Linker coupled to the polymer through at least a portion of the amino groups;
contacting the solid support having a Linker with a series of amino acid residues under conditions conducive to the formation of a specified amino acid sequence coupled to the EH group of the Linker to form a linear peptidic sequence bound to a solid support.

24. A method for the preparation of a linear peptidic sequence bound to a solid support, the method comprising the steps of:
(a) providing a solid support according to claim 1 which comprises a solid particle having a plurality of amino groups and at least one Linker coupled to the solid particle through at one of the amino groups;
(b) contacting a N-protected amino acid residue with the solid support under conditions conducive to coupling the free-carboxylate of the amino acid with at least a portion of the EH residues of the polymer support to form an ester or thioester bond;
(c) contacting the solid support having a N-protected amino acid coupled thereto with a combination of chemicals suitable for the deprotection of the N-protected amino-acid group;
(d) contacting a N-protected amino acid residue with the solid support having an amino acid sequence coupled to the Linker under conditions conducive to formation of an amide bond to extend the amino acid sequence by one residue;
(e) contacting the polymer solid support having an amino acid sequence coupled thereto with a combination of chemicals suitable for the deprotection of the protected N-terminal amino group; and
(f) repeating steps (d) and (e) to synthesize a specified amino acid coupled to the EH group of the Linker to form a linear peptidic sequence bound to a polymer support.

25. A method for the preparation of a linear hybrid substrate having peptide and polyketide residues, the method comprising the steps of:
providing a solid support according to claim 1; and
contacting the solid support according to Formula I with a series of amino acid residues and polyketide residues under conditions conducive to the formation of a specified hybrid peptide/polyketide sequence coupled to the EH group of the Linker to form a linear hybrid substrate sequence bound to the polymer support.

26. A method for the preparation of macrocyclic molecules comprising:
providing a substrate comprising an activated acyl residue and a pendant nucleophile separated by a linear backbone where the activated acyl residue is coupled to a solid support where the substrate is represented by Formula V: 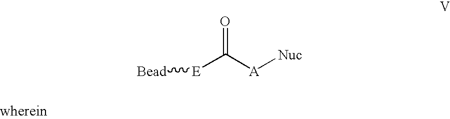
A is a peptidic sequence, synthetic hydrocarbon group, a polyketide, —(CH₂CH₂O)ₛ—, or a combination thereof having at least about 10 atoms in a linear backbone connecting the activated acyl, —C(O)E˜˜Polymer group and the Nuc group;
s is an integer from 1 to about 10;
Nuc is NH₂ or OH; and
Bead [symbol] E is a solid particle according to claim 1; and
contacting a purified TE domain protein with the solid support bound substrate under conditions conducive to formation of an transient TE-O-acyl bond and subsequent formation of a macrocyclic product by displacement of a TE domain by a pendant nucleophile.

27. The method of claim 26, wherein the group A comprises at least one peptidic sequence, at least one polyketide sequence, and optionally one or more groups selected from a synthetic hydrocarbon group, —(CH₂CH₂O)ₛ—, or a combination thereof such that A has at least about 10 atoms in a linear backbone.

28. A macrocyclization method of claims 26, according to Formula VI: 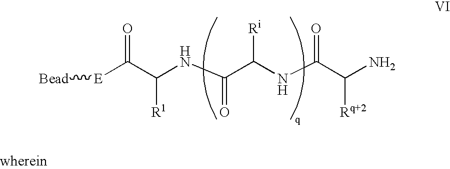
Rⁱ are independently selected for each value of i from 1 to q+2 from the group consisting of hydrogen, synthetic and biosynthetic amino acid residue side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where
each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol;
q is an integer from about 3 to about 22; and
Bead [symbol] E is a solid support according to claim 1.

29. A macrocyclization method of claim 26 where the macrocyclization substrate is a substrate according to the Formula VII: 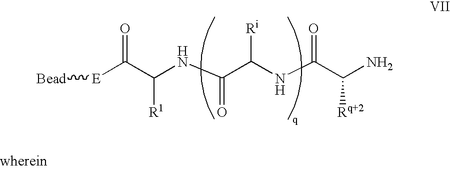
Rⁱ are independently selected for each value of i from 1 to q+2 from the group consisting of hydrogen, synthetic and biosynthetic amino acid residue side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where
each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol;
the amino acid residue bearing the R^{q+2} side chain had stereochemistry as shown in Formula VII;
q is an integer from about 3 to about 22; and
Bead [symbol] E is a solid support according to claims 1.

30. A macrocyclization method claims 26 wherein the group A comprises at least one peptidic sequence, at least one polyketide sequence, and optionally one or more groups selected from a synthetic hydrocarbon group, —(CH₂CH₂O)ₛ—, or a combination thereof such that A has at least about 10 atoms in a linear backbone.

31. A macrocyclization method claims 26 wherein:
Nuc is OH; and
A is a polyketide or a hybrid polyketide-peptide group, which optionally has one or more hydrocarbon or oxyalkylene groups, and which has from about 10 to 40 atoms in a linear backbone of A;

32. A macrocyclization method according to claim 26 where the macrocyclization substrate is a substrate according to the Formula VIII: 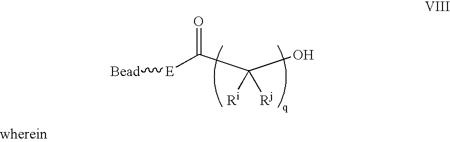
Rⁱ is independently selected for each value of i from 1 to q from the group consisting of hydrogen, C₁₋₆alkyl, hydroxy, halogen, C₁₋₆alkoxy;
R^{j} is independently selected for each value of j from 1 to q from the group consisting of hydrogen, C₁₋₆alkyl; or
CRⁱR^{j}, taken in combination, is a keto group;
where carbon having inequivalent Rⁱ and R^{j} groups for each i between 1 and q inclusive may be a (R), (S) or racemic stereocenter; and
q is an integer from about 10 to about 24.

33. A macrocyclization method of claims 26, wherein the substrate is represented by Formula IX: 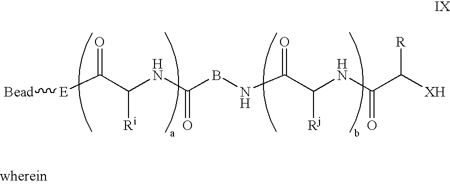
B is a polyketide residue having between about 2 and about 12 carbon atoms in a linear chain which may include one or more amide linkages or double bonds in the linear chain and which may be optionally substituted with between one and 12 groups selected from C₁₋₆alkyl, C₂₋₆alkenyl, C₂₋₆alkynyl, C₃₋₈cycloalkyl, hydroxy, or C₁₋₆alkoxy;
R, Rⁱ, and R^{j} are independently selected for each value of i from 1 to a and for each value of j from 1 to b from the group consisting of hydrogen, synthetic and biosynthetic amino acid residue side chains including side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where
each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol;
a and b are independently selected integers from about 2 to about 10;
X is O or NH; and
Bead [symbol] E is a solid support as defined in claim 1.

34. A macrocyclization method of claim 26 where the macrocyclization substrate is a substrate according to Formula X: 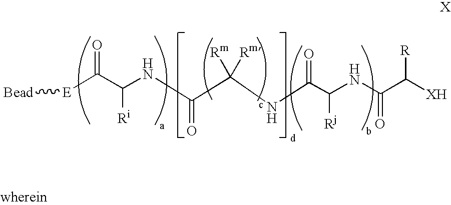
R, Rⁱ, and R^{j} are independently selected for each value of i from 1 to a and for each value of j from 1 to b from the group consisting of hydrogen, synthetic and biosynthetic amino acid residue side chains including side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where
each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol;
R^{m} and R^{m}′ are independently selected for each value of m from 1 to m from the group consisting of hydrogen, C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, and hydroxyC₁₋₆alkyl, or
R^{m} and R^{m}′ taken in combination form a keto group, or
adjacent (CR^{m}R^{m}′) residues, taken in combination, form an optionally substituted 1,2-vinylidene group;
a and b are independently selected integers from about 2 to about 10;
c is an integer of from about 2 to about 12;
d is an integer of from about 1 to about 5;
X is O or NH; and
Bead [symbol] E is a solid support as defined in claim 1.

35. A macrocyclization method according to claim 26 wherein each of the d residues in Formula X according to the formula: 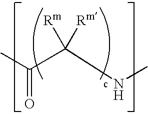
are independently selected from groups according to Formula XI: 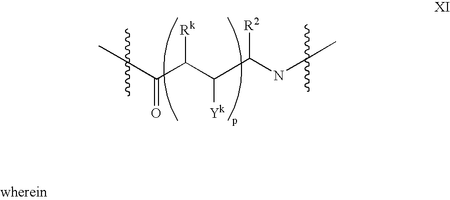
R² and each occurrence of R^{k} for each value of k from 1 to p are independently selected from the group consisting of hydrogen, C₁₋₆alkyl, C₂₋₆alkenyl, C₂₋₆alkynyl, C₃₋₈cycloalkyl, C₇₋₁₂aralkyl, hydroxy, C₁₋₆alkoxy, and hydroxyC₁₋₆alkyl;
Y is independently selected for each value of k from 1 to p from the group selected from hydrogen, hydroxy, C₁₋₆alkoxy, C₁₋₆alkyl, or keto; or
—(CHR^{k}CHY)— taken in combination form a 1,2-vinylidene group; and p is an integer of from about 1 to about 5.

36. A method of formation of a library of macrocyclic molecules comprising:
providing a plurality of solid support bound substrates suitable for macrocyclization according to Formula V: 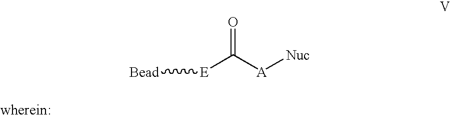
A is a peptidic sequence, synthetic hydrocarbon group, a polyketide, —(CH₂CH₂O)ₛ—, or a combination thereof having at least about 10 atoms in a linear backbone;
s is an integer from 1 to about 10;
Nuc is NH₂ or OH;
Bead [symbol] E is a solid support having at least one amino functional group;
where each solid support substrate of the library of solid support bound substrates comprises a chemically distinct combination of A and Nuc; and
contacting purified excised TE domain protein with each solid support bound substrate having a chemically distinct combination of A and Nuc under conditions conducive to formation of an transient TE-O-acyl bond and subsequent formation of a macrocyclic product by displacement of a TE domain by a pendant nucleophile, Nuc such that a plurality of chemically distinct macrocycles are formed.

37. A method of claim 36, wherein each solid support bound substrate having a distinct A and Nuc combination is physically segregated from other solid support bound substrates of the library of solid support bound substrates such that chemically distinct macrocycles of the library are segregated after TE domain catalyzed macrocyclization.

38. A method of claim 36, wherein the group A comprises at least one peptidic sequence, at least one polyketide sequence, and optionally one or more groups selected from a synthetic hydrocarbon group, —(CH₂CH₂O)ₛ—, or a combination thereof such that A has at least about 10 atoms in a linear backbone.

39. A method of claim 36, wherein the library of solid support bound substrates are substrates according to Formula VI: 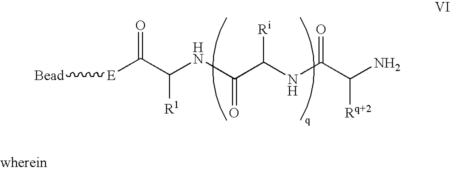
Rⁱ are independently selected for each value of i from 1 to q+2 from the group consisting of hydrogen, synthetic and biosynthetic amino acid residue side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where
each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol;
q is an integer from about 3 to about 22;
Bead is cross-linked polyethylene glycol acrylamide resin;
at least one amino acid residue in the peptidic sequence has a constant composition and stereochemistry for each substrate of the invention; and
each solid support bound substrate of the library has a distinct combination of stereochemistry or set of Rⁱ groups when i is varied from 1 to q+2 such that each solid support bound substrate of the library has a chemically distinct peptidic sequence.

40. A method of claim 36, wherein the library of solid support bound substrates are substrates according to Formula IX: 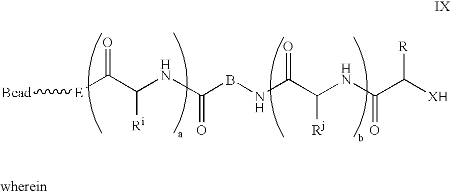
B is a polyketide residue having between about 2 and about 12 carbon atoms in a linear chain which may include one or more amide linkages or double bonds in the linear chain and which may be optionally substituted with between one and 12 groups selected from C₁₋₆alkyl, C₂₋₆alkenyl, C₂₋₆alkynyl, C₃₋₈cycloalkyl, hydroxy, or C₁₋₆alkoxy;
R, Rⁱ, and R^{j} are independently selected for each value of i from 1 to a and for each value of j from 1 to b from the group consisting of hydrogen, synthetic and biosynthetic amino acid residue side chains including side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where
each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol;
a and b are independently selected integers from about 2 to about 10;
X is O or NH; and
Bead [symbol] E is a solid support as defined in claim 1;
at least one amino acid residue in the peptidic sequence has a constant composition and stereochemistry for each substrate of the invention; and
each solid support bound substrate of the library has a distinct combination of stereochemistry or set of amino acid and polyketide residues such that each solid support bound substrate of the library has a chemically distinct hybrid substrate.

41. A macrocycle according to Formula XIII: 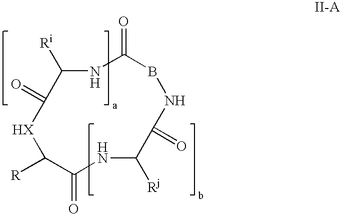
wherein
B is a polyketide residue having between about 2 and about 12 carbon atoms in a linear chain which may include one or more amide linkages or double bonds in the linear chain and which may be optionally substituted with between one and 12 groups selected from C₁₋₆alkyl, C₂₋₆alkenyl, C₂₋₆alkynyl, C₃₋₈cycloalkyl, hydroxy, or C₁₋₆alkoxy;
R, Rⁱ, and R^{j} are independently selected for each value of i from 1 to a and for each value of j from 1 to b from the group consisting of hydrogen, synthetic and biosynthetic amino acid residue side chains including side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where
each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol;
a and b are independently selected integers from about 2 to about 10; and
X is O or NH.

42. A macrocycle of claim 41, wherein the macrocycle is a compound of Formula XIV: 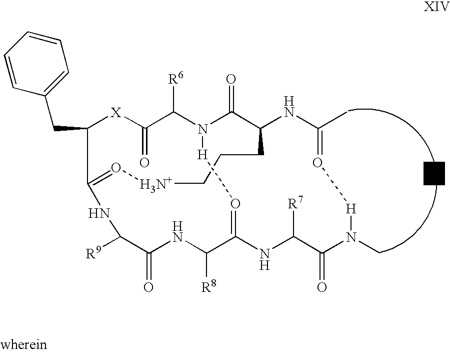
B is a polyketide residue having between about 2 and about 12 carbon atoms in a linear chain which may include one or more amide linkages or double bonds in the linear chain and which may be optionally substituted with between one and 12 groups selected from C₁₋₆alkyl, C₂₋₆alkenyl, C₂₋₆alkynyl, C₃₋₈cycloalkyl, hydroxy, or C₁₋₆alkoxy;
R⁶, R⁷, R⁸, and R⁹ are independently selected from the group consisting of synthetic and biosynthetic amino acid residue side chains including side chains selected from C₁₋₆alkyl, C₂₋₆alkenyl, C₂₋₆alkynyl, C₃₋₈cycloalkyl, C₃₋₈cycloalkylC₁₋₄alkyl, hydroxy, C₁₋₆alkoxy, hydroxyC₁₋₆alkyl, thioC₁₋₆alkyl, amino, mono and di(C₁₋₆alkyl)amino, saturated, partially unsaturated or aromatic heterocyclic groups having 5 to about 15 ring atoms, 1 to about 3 rings and 1, 2, or 3 N, O or S ring atoms, aryl, arylmethyl, and heteroarylmethyl, where each amino acid residue side chain may be optionally substituted with one or more substituents selected from the group consisting of hydrogen, chloro, fluoro, bromo, iodo, cyano, nitro, amino, hydroxy, C₁₋₆alkyl, aryl, benzyl, carboxylate, carbamoyl, thiol, C₁₋₆alkylthiol; and
X is O or NH.

43. A substrate for hydrolysis by fatty acid synthase according to Formula XV: 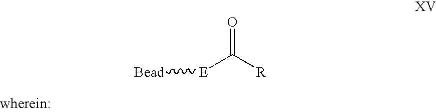
R is a fatty acid hydrophobic residue having between about 4 and about 36 carbon atoms; and
Bead [symbol] E is a solid support according to claim 1.

44. A method for determining enzymatic selectivity of an enzyme comprising
providing a library of solid support bound substrates according to claim 9, wherein each solid support bound substrate of the library has a distinct chemical composition;
contacting each solid support bound substrate of the library sequentially, in parallel, or in combination with the enzyme; and
determining analytically or spectroscopically, the substrates of the library which undergo reaction with the enzyme.

45. A method for determining enzymatic selectivity of an enzyme comprising
providing a library of solid support bound substrates according to claim 43, wherein each solid support bound substrate of the library has a distinct chemical composition;
contacting each solid support bound substrate of the library sequentially, in parallel, or in combination with the enzyme; and
determining analytically or spectroscopically, the substrates of the library which undergo reaction with the enzyme.
